(19) United States
(12) Patent Application Publication
(10) Pub. No.: US 2026/0215679 A1
(43) Pub. Date: Jul. 30, 2026

(54) METHOD AND APPARATUS FOR PERFORMING EYE TESTS AND SELLING EYEGLASSES

(71) Applicant: VMAX VISION, Maitland, FL (US)

(72) Inventor: Shui T LAI, Windermere, FL (US)

(73) Assignee: VMAX VISION, Maitland, FL (US)

(21) Appl. No.: 19/293,542

(22) Filed: Aug. 7, 2025

Related U.S. Application Data
(60) Provisional application No. 63/750,445, filed on Jan. 28, 2025.
(60) Provisional application No. 63/763,724, filed on Feb. 26, 2025.

(51) Int. Cl. A61B 3/04; A61B 3/00; A61B 3/032; A61B 3/11; A61B 3/18; A61B 5/00; A61B 5/107; G06Q 30/0601; G06T 7/50; G06T 7/62; G06T 11/60; G16H 80/00

(52) U.S. Cl. CPC A61B 3/04; A61B 3/0058; A61B 3/0075; A61B 3/0091; A61B 3/032; A61B 3/0325; A61B 3/111; A61B 3/18; A61B 5/1072; A61B 5/741; G06Q 30/06432; G06T 7/50; G06T 7/62; G06T 11/60; G16H 80/00; A61B 2560/0238; G06T 2200/24; G06T 2207/10016; G06T 2207/20092; G06T 2207/20212; G06T 2207/30041; G06T 2207/30201; G06T 2207/30204

(57) ABSTRACT

Self-service optical stations having a form factor of a kiosk perform essential business transactions of a traditional optical store. A user takes eye tests and buys eyeglasses at the station without an attendant. The user may be sitting or standing in front of the station while conducting eye tests and business transactions. A telemedicine module communicates refraction results and patients in-take form to a refractionist at a remote location for review and approval. A virtual try-on module assists users to select matching frames to the user's face shape and lifestyle. A measurement module measures frame fitting parameters including pupil distance and the segment height while the user virtually wears a virtual frame. A point-of-sale module collects payment; a lab order module sends the purchase order to the fabrication facility where the lenses may be fabricated and assembled into the selected frames.

### CROSS-REFERENCE TO RELATED APPLICATIONS

This specification claims priority to U.S. Provisional Application, Ser. No. 63/750,445 (Docket #VMAX-001-PROV), entitled “METHOD AND APPARATUS FOR PERFORMING SELF SERVICE OPTICAL EXAMINATIONS,” filed Jan. 28, 2025, Shui T. Lai, and U.S. Provisional Application, Ser. No. 63/763,724 (Docket #VMAX-001-PROV-2), entitled “SELF SERVICE OPTICAL STATIONS,” filed Feb. 26, 2025, Shui T. Lai, both of which are incorporated herein by reference.

### BACKGROUND

### (1) Technical Field

This disclosure relates to optical examinations (“exams”) and methods for selling eyeglasses. More particularly, this disclosure relates to self-service workstations for performing optical exams and selling eyeglasses without third party assistance and to methods for performing such optical exams and selling eyeglasses.

### (2) Background

It is common today for a user that wishes to buy a pair of eyeglasses to go to an optical store, the office of an optometrist or the office of an ophthalmologist. In many cases, such a person will need to update their eyeglass prescription. Measuring a person's eyes for a prescription for corrective lenses is referred to as performing a refraction procedure. Typically, a refraction procedure requires a refraction room. It is common for such refraction rooms to be relatively large. In many cases, the refraction room is approximately 15 ft long. In addition, the process requires a refractionist to operate a phoropter (i.e., the instrument that commonly performs measurements of the eyes). In many such cases, the user will also need to purchase an eyeglass frame. Typically, the service provider will need to carry a wide selection of frames from which the user can select one they like. Thus, the service provider will need to have several racks of frames on display.

Today, all these activities are typically done in a brick-and-mortar facility having a service area of about 1500 or more square feet. In addition to the facility, several people are typically employed to perform various duties necessary to service users seeking such services, including performing the refraction, selling and fitting the eyeglasses, conducting business transactions, delivering of the eyeglasses to the user, and managing the store. Accordingly, the operating costs for running an optical store are significant. In addition to rent and salaries, the cost of establishing and maintaining a large inventory of frames and setting up and maintaining a frame display adds to the overall operating costs. Hence, it is desirable to reduce the operating costs by reducing the space requirement, the people requirement, and the frame inventory requirement.

### SUMMARY OF THE INVENTION

A self-service optical station is disclosed herein. In some embodiments, the self-service optical station is a compact structure that houses various features and processes that are integrated into the station to provide a self-service optical transaction to a user. The compact structure is the home base of the self-service optical workstation (hereafter the “station”). Accordingly, the station functions as a standalone self-sufficient unit that offers a self-service optical transaction to a user, such as performing an eye test, selling eyeglasses, making measurements related to fitting the eye-frames, taking payments, and performing other services that are currently offered at a brick-and-mortar store.

The station comprises a self-service refraction instrument, such as a self-refractor, capable of performing a subjective self-refraction procedure and providing a user with an eyeglass prescription from which the user can order eyeglasses, contact lenses, or eye surgery. In some embodiments, the self-service optical transaction performed by the station includes the following:
a subjective self-refraction procedure to attain a prescription for corrective lenses;
a “frame try-on” function that allows the user to virtually try on one or more frames (in some embodiments, the frames in the try-on process are virtual frames);
a measurement function that measures frame characteristics and features of the user that are required to fabricate a pair of eyeglasses for the user, including measuring the user's pupil distance and the appropriate segment height;
a point-of-sale function that allows the user to make payments and process such payments;
an optical laboratory function, including an ordering function that places an order for a pair of eyeglasses for the user using the prescription from the self-refraction function and the frame selected through the frame try-on function;

In some embodiments, the station interacts with other remote services over a telemedicine platform. For the purposes of this disclosure, a telemedicine platform refers to infrastructure, including communications infrastructure, services, and support that enable private, secure, HIPAA (Health Insurance Portability and Accountability Act) compliant communication with a health care professional. In some such embodiments, the platform comprises a communication link to connect the self-refractor to a qualified refractionist at a remote location (a refractionist can be a qualified professional, such as an optometrist, ophthalmologist or other healthcare provider trained to use a refractor). In some embodiments, the subjective self-refraction procedure results can be reviewed by the refractionist in real-time (i.e., synchronously). Alternatively, review can be performed later when review is more convenient for a refractionist (i.e., asynchronously).

In some embodiments, the user can interact directly with the station through at least one user interface device to: (1) access the on-station self-refractor to obtain an eyeglass prescription, (2) try on one or more pairs of virtual frames, (3) select the frames to be purchased, (4) measure at least some frame parameters of the frame that was selected, (5) generate a fabrication order, (6) pay for the exam and the purchase of one or more pairs of eyeglasses, and (7) have the eyeglasses that were made from the fabricating order delivered to a selected delivery address.

Also disclosed is a highly efficient business method for providing optical services through a kiosk. For the purpose of this disclosure, a kiosk is a small, structure from which optical services are issued to a user. In particular, the kiosk is only as large as is required to house the equipment other modules and services of the disclosed apparatus. In accordance with some embodiments of the disclosed method, substantial cost and time savings for users result from: (1) enabling technologies, (2) eliminating operators, attendants and several manual methods; (3) replacing operators, attendants and the need for performing manual methods through computer automation, and (4) time-sharing of a refractionist. Enabling timing-sharing of the refractionist allows that refractionist to oversee multiple stations, thus eliminating the need for a doctor or other costly employees at each optical store.

Embodiments of the presently disclosed method and apparatus can also include a Virtual Try-On Module (VTM). In some embodiments, images of frames in the form of 3D models are included in the VTM. During a frame virtual try on, the function combines the face image of a user with images of one of a series of virtual frames, thereby the combined image appears that the user is wearing those frames on its face (i.e., is virtually wearing the frames). The try on function eliminates entirely the need to maintain a physical inventory of frames, another huge cost saving.

In some embodiments, the cost of constructing a standalone workstation is much less than the startup costs of opening an optical store. In one embodiment, the cost of starting and running a brick-and-mortar optical store is about 10 times the cost of building and running a self-service optical station. Instead of having one store, it is possible to construct 10 stations and spread them across a region. This offers substantially more convenience and access to users.

In some embodiments, a self-service optical station is a kiosk comprising:
an enclosure;
a pedestal;
a self-refractor housed within the enclosure; and
an eyepiece optically coupled to the self-refractor providing an optical entrance for a user's eye;
an Elevation Module;
   wherein the self-refractor is coupled to the Elevation Module, the height of the refractor is adjustable inside the enclosure;
   wherein the range of the refractor's travel is from 38 inches to 96 inches above the floor, to accommodate a user either sitting in a chair, or standing in front of the kiosk structure;
   wherein the Elevation Module moves the eye piece of the refractor to the eye level of the user; and
   wherein the user performs a self service subjective refraction using the refractor.

In some embodiments, a method is disclosed for enabling a user to purchase a pair of eyeglasses without third party assistance, in which a user performs the following;
a) walking up to a self-service optical station;
b) interacting with at least one user interface device, such as Touching a screen on the unit to initiate the procedure;
c) providing user information, including contact information and other personal information, such as the height of the user, through one or more interface devices;
d) moving the self-refractor to the eye level of the user;
e) performing a subjective self-refraction procedure in which a user follows either audible (i.e., voice) instructions from one or more of the user interface devices of the station or instructions displayed by the station through one or more of the user interface devices and provides instructions and/or further information in response to the audible instructions;
f) trying on a virtual frame;
g) selecting one or more frames to purchase;
h) selecting lenses;
i) making a payment; and
j) receiving a finished pair of eyeglasses.

Also disclosed is a VTM, comprising:
a module to facilitate integrating an image of virtual frames with the image of a user, the composite image showing the user virtually wearing the virtual frame as if wearing an actual physical frame;
a Face Shape Analyzer Module, in some embodiments determines characteristics of the user's face. A Measurement Module makes measurements of frame parameters on the virtual frame. It also measures the pipul distance and the segment height while the user virtually wears the virtual frame;
a Frame Selection Module facilitates selection of a subset of frames, frames that suited to the user, based on user's face shape and lifestyle. Based on rules governing which frame shapes are best suited to the user's face shape, the frame selection module implements rules governing which frame shapes best suited for the user's lifestyle;
a frame selection module selects a set of virtual frames from a frame database that stores an inventory of virtual frames. Furthermore, the database stores user information, including user face shapes, lifestyle choices, and a history of user purchases. In some embodiments, marketing promotions are constructed from information collected on the user face shapes, lifestyle choices and past purchase history. In some such embodiments, the promotion includes launching a new line of frame style or frames the users had considered purchasing at some point in the past, but did not make the purchase, among many other marketing uses.

In some embodiments, the Frame Selection Module selects a first set of frame images from the frame database based on the shape of the user's face. In some such embodiments, the Frame Selection Module selects a second set of frame images based on the user's lifestyle information retrieved from the user database; thereby both first and second sets of selected frames, or the subset where the two sets of frames overlap, are presented to the user to try on by the VTM.

In some embodiments, the device for virtual frame try-on interacts with user interface devices, such as a camera capturing an image of the user and a display monitor showing the composite image of the user virtually wearing a virtual frame.

The station also provides a movable platform, part of an Elevation Module, to move the camera and self-refractor to a desired height relative to the user's eye. In some embodiments, the Measurement Module establish a calibration factor, that is used to measure the dimensions of an object in the image captured by the camera in the VTM,

A method of selling eyeglasses is also disclosed, some embodiments of which comprise;
having an inventory of virtual frames accessible to a user;
capturing a user's image;
determining the face shape of the user;
inquiring about the lifestyle of the user;
recommending frames from the virtual frame inventory based on the user's face shape and/or lifestyle;
trying on virtual frames;
measuring a pupil distance and segment height of the user while the user is virtually wearing the virtual frames; and
selecting frames to purchase from the inventory of virtual frames.

Also disclosed herein is a user database comprising information of a user, the information including one more of the following: 1) face shape; 2) lifestyle choices; 3) history of previous frame purchases; 4) user's mailing address; 5) user's phone number; and 6) user's email address.

Also disclosed is a comprehensive eye exam method and device that is more efficient, lower operating costs, and offer more convenience for the user by adding more opening hours. Furthermore, it satisfies regulatory standards imposed by optometric authority and certain state legislation in the US. In some embodiments, a self-refractor is used instead of a phoropter, thereby the user performs subjective self-refraction without a doctor or an assistant. This reduces the time of performing the refraction by a doctor. Additionally, finalizing steps of the subjective refraction by a doctor is to be carried out in a video conference instead of in person. This improves efficiency by time-sharing a doctor to multiple optical locations though telemedicine, instead of having one doctor at each location, or having the doctor to travel from office to office.

Also disclosed is a method for promoting sales of eyeglasses by recommending frames to a user through email and digital media, the digital media including Face book, YouTube, Twitter/X, Instagram, and blogging, wherein the frames are selected based on the user's face shape and/or lifestyle and the frames are selected from the frame inventory.

### BRIEF DESCRIPTION OF THE DRAWINGS

The disclosed method and apparatus, in accordance with one or more various embodiments, is described with reference to the following figures. The drawings are provided for purposes of illustration only and merely depict examples of some embodiments of the disclosed method and apparatus. These drawings are provided to facilitate the reader's understanding of the disclosed method and apparatus. They should not be considered to limit the breadth, scope, or applicability of the claimed invention. It should be noted that for clarity and ease of illustration these drawings are not necessarily made to scale.

FIG. 1 is a simplified diagram of a station in accordance with one embodiment of the disclosed method and apparatus

FIG. 2 is a simplified diagram of another station in accordance with another embodiment of the disclosed method and apparatus.

FIG. 3 is a simplified illustration of a portion of a station, showing a user interface plate in accordance with some embodiments of the presently disclosed method and apparatus.

FIG. 4 is an illustration of one embodiment of a control unit in accordance with one embodiment of the station, as well as some of the components coupled to the control unit.

FIG. 5 is a more detailed illustration of the modules within the System Processor in accordance with one embodiment of the station.

FIG. 6 is a simplified schematic of components that are used to interface with the Refraction Control Module.

FIG. 7 is an illustration of part of the station showing the Telemedicine Module and the associated components that interact with the Telemedicine Module.

FIG. 8 is a simplified schematics of the steps of a comprehensive eye exam.

FIG. 9 is a simplified schematic of the Virtual Try-On Module (VTM) and various components that are used to interface with the VTM.

FIG. 10 is an illustration of a reference image used in a measurement calibration procedure.

FIG. 11 is an illustration of a composite image showing a user virtually wearing a virtual frame.

FIG. 12 is an illustration of the Lab Order Module and some of the elements of some embodiments of the disclosed station related to the Lab Order Module.

FIG. 13 is a flowchart of one embodiment of a method for performing a self-service optical transaction in accordance with the present invention.

FIG. 14 is a flowchart of one embodiment of a subjective self-refraction procedure.

The figures are not intended to be exhaustive or to limit the claimed invention to the precise form disclosed. It should be understood that the disclosed method and apparatus can be practiced with modification and alteration, and that the invention should be limited only by the claims and the equivalents thereof.

### DETAILED DESCRIPTION

A method and an apparatus for performing self-service optical transactions are disclosed. The method allows a user to approach a self-service optical station (alternatively referred to as an optical kiosk and hereafter referred to as the “station”) to receive services, including having the station perform subjective self-refraction, make measurements of the user's pupil distance and segment height, allow the user to virtually try on glasses, transact a sale and collect payment. Those of ordinary skill in the art will understand that subjective refraction requires the user to provide feedback to determine which lens provides the best correction. This is as opposed to “auto-refraction”, in which the correction is determined without input from the user (i.e., the patient).

In some embodiments, operations such as making measurements and analyzing user information are performed as “internal operations”. Internal operations are defined as operations that are performed without the assistance of the user. In some embodiments, the user is unaware that such internal operations are being performed. In some embodiments, user information includes information regarding face shape, skin color, measurements of the pupil distance and various other required measurements.

Other types of user information and other services provided by the station are detailed further below.

### Compact Standalone Structure of the Station

Typical, refraction procedures are performed in an examination (“exam”) room 18 feet long. Reducing the amount of space required to perform the such procedures results in significant advantages and efficiencies. Furthermore, typical refraction procedures requires an operator who is either a refractionist or an optometrist. Learning the skills required to perform a refraction procedure takes months or even years before an operator succeeds in performing an effective subjective refraction.

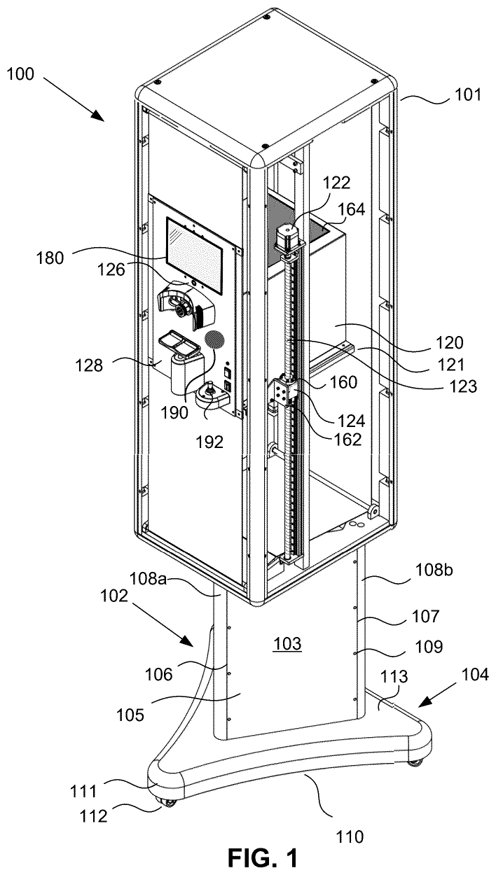

FIG. 1 is a simplified diagram of a self-service optical station 100 in accordance with one embodiment of the disclosed method and apparatus. In some embodiments, the station 100 integrates several technologies, fitting them in a compact structure. A self-service refraction instrument (“self-refractor”) 120 fits inside the station 100. Many benefits derive from the relatively small size of the station 100. In addition to performing self-refraction, several other services are also performed by station 100. Providing a compact structure and integrating several features into the station 100 reduce the cost of providing the services and so result in large cost and time savings for the users.

### Self-Refractor

In one embodiment of station 100 in accordance with the disclosed method and apparatus, an enclosure 101 is mounted on a pedestal 102. In some such embodiments, the pedestal 102 consists of an upper member 103 and a lower member 104. The upper member 103 has three essentially rectangular cover plates 105 (only one of which is visible in FIG. 1). Each side plate 105 is coupled to the other two side plates 105 along edges 106 by an interceding post 108a, 108b and secured using screws 109. A third post lies behind the upper member 103 and is not visible in FIG. 1.

The lower member 104 comprises an essentially triangular-shaped bottom plate with a top surface 113 sized to accept and support the upper member 103. The upper member 103 is coupled to the lower member 104 by lag bolts (not visible in FIG. 1) that pass through holes (not visible in FIG. 1) in the top surface of the lower member 104 and are captured and secured by nuts (not visible in FIG. 1) from the underside of the lower member 104. The upper member 103 has a top surface (not visible in FIG. 1) on which the bottom of the enclosure 101 rests. The enclosure 101 is secured to the upper member 103 by lag bolts similar to the manner in which the lower member 104 is secured to the upper member 103, with the lag bolts extending up from the upper member 103 through holes in the bottom of the enclosure 101 and secured by nuts (not visible in FIG. 1). The lower member 104 is bowed inward along a portion 110 of the lower member 104 between each of three vertices 111. The bowing of the portions 110 form a relatively acute angle at the vertices 111, making it easier for a user to approach the station 100, either on foot, in a wheelchair, or when using a walker or other aid. That is, the portion between the vertices 110 is carved out to allow a person to stand next to or to place the wheels of a wheelchair within the carved out area.

The upper member 103 of the pedestal 102 supports the enclosure 101 at a sufficient height to allow the legs of a user sitting in a wheelchair to slide under the enclosure 101. The front wheels of a wheelchair can straddle the vertices 110 of the triangular base, allowing a user confined to a wheelchair to sit relatively close to the station 100 and thus comfortably place their face close to an eyepiece 308. In some embodiments, caster wheels 112 beneath the lower member 104 allow it to move. In other embodiments, stationary feet are used in place of the caster wheels 112. In yet other embodiments, the lower member 104 rests directly on the ground or on a surface on which the station 100 is placed.

In some embodiments, the station 100 has a footprint in a range from 12 square feet to 100 square feet. In some embodiments, the footprint of the station 100 is approximately 4 ft. wide by 4 ft. deep (i.e., 16 square ft.), creating a small form factor. In other embodiments, the station 100 may have a larger footprint to 100 square foot. When placing the station 100 in a space for which rent is to be paid, having a small footprint reduces the rent compared to the relatively large space of a typical brick and mortar optical store (e.g., 2,000 or more square ft.). Furthermore, it can be challenging to find suitable space in a favorable location for a 2000 square ft. brick and mortar optical store. The small size also makes it easier to find rental spaces in which to locate the station 100 in locations such as, airports, college campuses, shopping malls, department stores, convention centers, grocery stores, pharmacy stores, and other locations having relatively high pedestrian traffic.

### Elevation Module

The self-refractor 120 is mounted on a moveable platform 121 within the enclosure 101. While the term “within the enclosure” is used herein, it should be clear that in some embodiments (not shown), either the self-refractor 120 or the platform 121 or both, partially protrude outside the walls of the enclosure 101. In some embodiments, the self-refractor 120 conforms to the refraction device disclosed in pending U.S. patent application Ser. No. 17/800,532 to Shui Lai (“the '532 application”). Alternatively, the self-refractor 120 conforms to the refraction device disclosed in U.S. Pat. No. 9,730,578, entitled “Self-Guided Subjective Refraction Instruments and Methods” (“the '578 patent”). Both the '532 application and the '587 patent are hereby incorporated by reference in the present specification. It should be noted that in some embodiments, the self-refractor may comprise any self-refractor instrument not expressly disclosed herein.

The platform 121 is raised or lowered by a drive screw motor 122 that turns a drive screw 123 that runs through a threaded bushing 160 within a motion block 124. The motion block 124 is securely attached to the platform 121 by an L-bracket 162. As the drive screw 123 turns, threads of the drive screw 123 interact with the threads of the bushing 160 to drive the motion block 124 up or down the drive screw 123. As the drive screw 123 rotates in a first direction, the platform 121 will move laterally upward within the enclosure 101. When the direction of rotation of the drive screw 123 reverses, the platform moves downward. The platform 121 allows the self-refractor 120 to be adjusted to a height that is comfortable for a user under control of the drive screw motor 122.

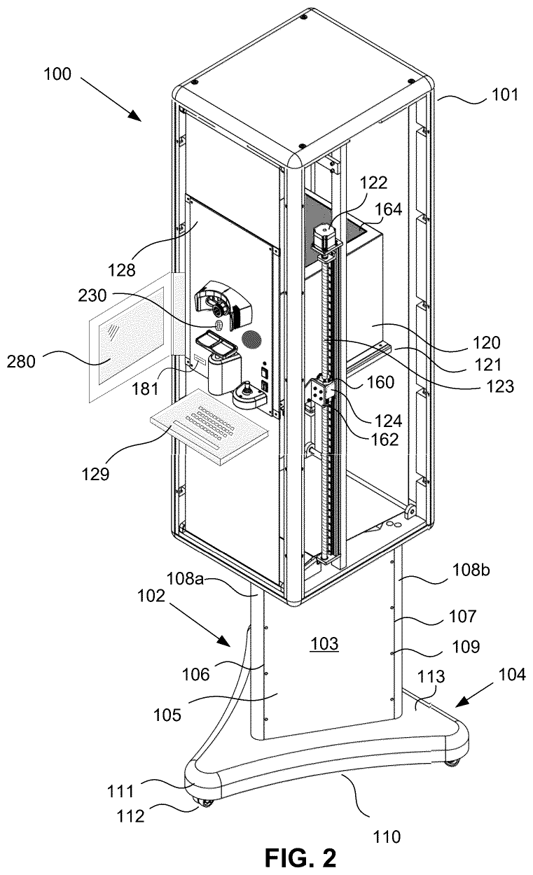

In some embodiments, the selected self-refractor 120 shown in FIGS. 1 and 2 fit inside the kiosk structure of the station 100. Since the self-refractor 120 is moveable, its size and weight are important considerations. If the self-refractor weighs over 100 Kg that would require substantially large and powerful motors in the Elevation Module. In some embodiments, the selected refractor in the present disclosure is a VASR manufactured by Vmax vision, Maitland FL. It is suitable for our requirements in terms of size and weight. The outside dimensions are 13 inches width×16 inches height by 22 inches depth. It weighs about 20 Kg. Two NEMA 23 stepper motors are sufficient to move the refractor sufficiently fast in the station.

In some embodiments, a control unit 164 coupled to the self-refractor 120. In some embodiments, the platform 121 has a range of motion that allows the platform 121 to be positioned anywhere within a range of about 30 inches. Motion of the platform 121 allows the station 100 to accommodate both a person having a height of 6 feet 6 inches and a person with a height of less than 4 feet. In some embodiments, the range of motion of the platform allows the station to accommodate a child of 40 inches. In other embodiments, the range of motion is depends upon the environment and the nature of the users and how the users use the station 100. More extended ranges can be constructed using a longer drive screw 123. Accordingly, people of various heights.

In some embodiments, the platform 121 moves the self-refractor 120 down to eye level of a person sitting in a wheelchair, allowing such wheelchair bound people to use the station 100. In addition, station 100 is configured to accommodate users, from 3 feet tall to 6 feet 6 inches, covering most of the population. In other embodiments, the platform 121 travel ranges can be expanded in a manner that remains within the scope of the disclosed method and apparatus.

The drive screw motor 122 is controlled by the control unit 164. In the embodiment shown in FIG. 1, the control unit 164 is mounted on top of the self-refactor 120. However, the placement of the control unit 164 depends upon the particular application and design of the station. In some embodiments, the control unit 164 is incorporated within the self-refractor 120. In other embodiments, the control unit 164 is mounted elsewhere within the station 100.

In some embodiments, a user interface plate 128 is mounted to the enclosure 101. The user interface plate 128 is mounted in a manner that makes components accessible to the user. Such components include user interface devices, such as a camera 126, a display screen 180, a printer 181, a speaker 190 and a joystick 192. In some embodiments, the display screen 180 is a touch sensitive screen. Accordingly, the touch sensitive screen can be used as a user input device to interact with the control unit 164. In some such embodiments, cutouts (not shown) in a front plate of the enclosure 101 expose components of the user interface plate 128. It should be noted that the front panel and side plates of the enclosure 101 are not shown in FIG. 1 in order to expose the components that lie behind the front plate and side plates.

FIG. 2 is a simplified diagram of a station 200 in accordance with another embodiment of the disclosed method and apparatus. The station 200 has several user interface devices, including a “flip screen”, such as a touch screen 280, a keyboard 229, a joystick, and microphone 230. It should be understood that both the station 100 and the station 200 can have other user interface devices not expressly disclosed herein. In other embodiments, user interface devices used to interact with the station 100, 200 include keypads, buttons, cameras and other user interface devices well known to those of skill in the art. Furthermore, in other embodiments, some of the user interface devices shown in FIGS. 1 and 2 are not provided. Furthermore, it should be understood that only a subset of all possible user interface devices are expressly disclosed for the sake of brevity.

In some embodiments, the touch screen 280 extends out from the user interface plate 128 to provide a user interface that allows the user to provide input to the system and the system to display information to the user.

In some embodiments, counterweights (not shown in FIG. 1 or 2) may be used to counterbalance the gravity pull of the self-refractor 120. In this configuration, a rope secures a weight that is about the weight of the self-refractor 120 to the station 100. The rope goes over a pulley attached near the top of the enclosure 101. Additional pulleys may be used to direct the rope. Such additional pulleys may be secured to convenient locations around the platform 121 which locations may be near the center of gravity of the self-refraction 120. The counterweights reduce the strain on the drive screw motor 122 when the drive screw motor 122 moves the platform 121 and the self-refractor unit 120 upward. That is, when gravity is pulling the weight down, the rope applies an upward force on the platform 121. Accordingly, the counterweight helps offset the effect of gravity on the platform 121, resulting in a relatively small residual resistance to the upward motion of the platform 121 (i.e., the difference between the weight of the counterweight and the weight of the platform 121 and the self-refractor 120), thus reducing the amount of force/torque needed to be provided by the drive screw motor 122.

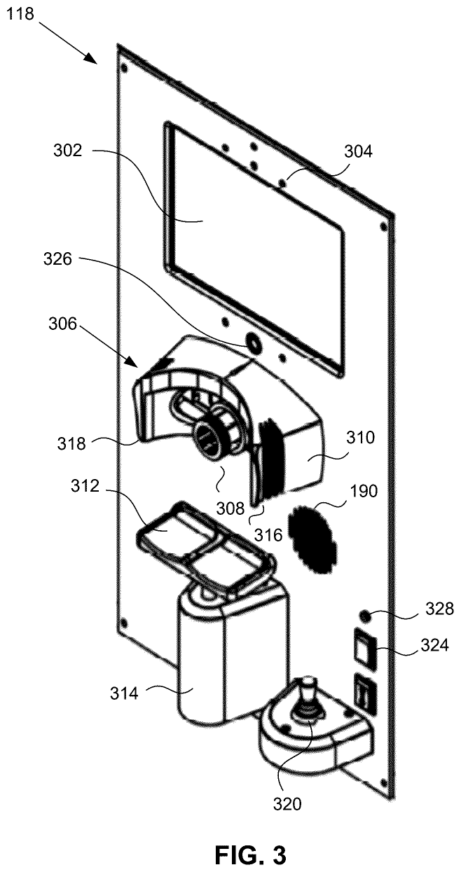

FIG. 3 is a simplified illustration of a user interface plate 128 in accordance with some embodiments of the presently disclosed method and apparatus. A cutout 302 in the user interface plate 128 allows the user to see the display screen 180 (see FIG. 1) mounted on the user interface plate 128. A refractor has an eye portal 306, such as an eye piece 308. It is optically coupled to the self-refractor 120. The self-refractor 120 can take measurements of the eye when the user places his eye on the eye piece. The eye piece 308 is the optical entrance of the self-refractor 120. In other embodiments, the eye portal is any opening optically coupled to the self-refractor 120, such that the self-refractor 120 has access to the user's eye. In some embodiments, the refractor user interface unit 306 also has a molded headrest 310 to help place the user's head in a position that aligns the user's eye with the eye piece 308. In some embodiments, the head rest 310 also functions as a face mask, to help block out stray light from entering the self-refractor 120.

In some embodiments, a moveable chinrest 312 provides a place for the user's chin to reside. In some such embodiments, the chinrest 312 moves to properly position the user's head and eye with respect to the eye piece 308 and the head rest 310. In some embodiments, the chinrest 312 is manually adjusted by the user using a height adjustment switch 324 to control the height of the chin rest 312. In such embodiments, the user's chin resides on the chin rest 312 and the height adjustment switch 324 is activated to move the chin rest 312 up or down to align the user's eye into the proper alignment with the eye piece. Properly aligning the user's eye with the eye piece maximizes the likelihood that an accurate eye image will be successfully captured. In some embodiments, such images are used for wavefront analysis during auto-refraction. In some embodiments, motors control the position of the headrest 310 and chinrest 312. Together with positioning sensors (not shown), the motors move the headrest 310 and chinrest 312 to align the user's eye with the eye piece 308 when the user makes initial contact with either the headrest 310, the chinrest 312 or both. In some embodiments, a camera hole 326 is located slightly above the eye piece 308. The camera 126 (see FIG. 1) is located behind the user interface plate 128. In other embodiments, the camera 126 is located at other convenient locations.

### Control Unit

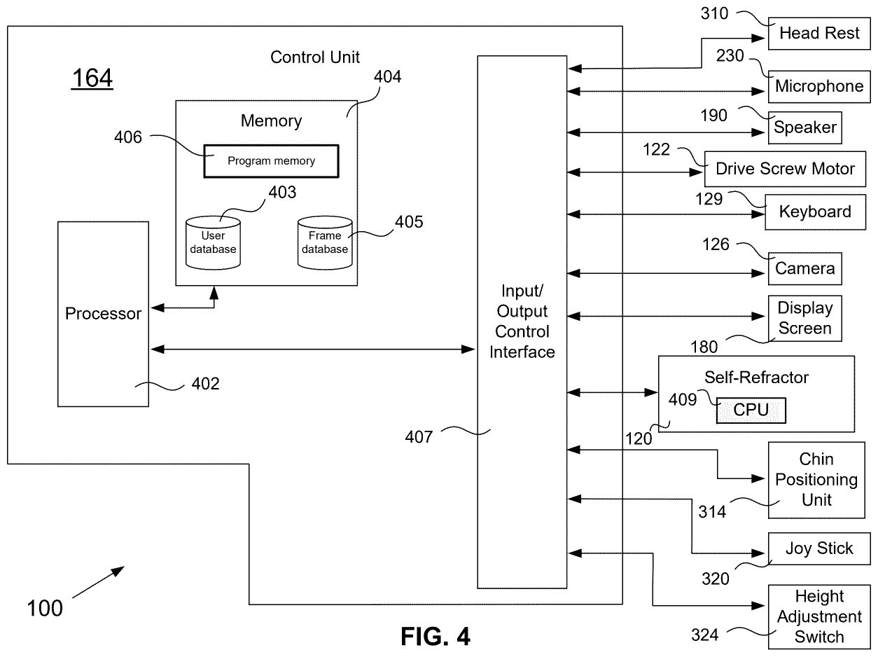

FIG. 4 is an illustration of one embodiment of a control unit 164 in accordance with one embodiment of the station 100, as well as some of the components coupled to the control unit 164. A System Processor 402 is coupled to a memory unit 404 and to an Input/Output Control Interface (IOCI) 407. In some embodiments, the System Processor 402 is a control processor, microprocessor, state machine, artificial intelligence engine, or other controller capable of receiving input and responding with appropriate control signals. In some embodiments, the System Processor 402 generates instructions for the user. In some such embodiments, instructions are in the form of text on the display screen, or computerized voice instructions during a subjective self-refraction procedure. In some embodiments, the system processor is one device. In other embodiments, the System Processor 402 may be several discrete devices that work together to form the System Processor 402. In some such embodiments, these discrete devices may be different from one another. For example, in some embodiments, the System Processor 402 may have a discrete state machine that performs one or more functions, an AI engine that performs other functions, and a programmable computer that performs still other functions. In some embodiments, these discrete devices, that comprise the System Processor 402, communicate with one another. In other embodiments, one or more of the discrete devices work completely independently of the others.

The System Processor 402 stores and accesses user information and frame information in a memory unit 404. A user database 403 and a frame database 405 are maintained within the memory unit 404. In other embodiments, such information is stored in a discrete memory unit (not shown) that is independent of the memory unit 404. In some such embodiments, the discrete memory unit resides somewhere within the station 100. In other embodiments, the discrete memory unit resides remotely, such as in a cloud server coupled to the control unit 164 by a communication link, such as a wireless internet connection. In some such embodiments, communication with the discrete memory unit is through the IOCI 407.

In some embodiments, the IOCI 407 serves as an interface between the System Processor 402 and each of the devices, including one or more user interface devices with which the System Processor 402 communicates. In other embodiments, the System Processor 402 is coupled directly to the memory 404 and to the devices.

In some embodiments, the interface devices include the: a microphone 328, a speaker 322, a keyboard 129, a camera 126, a display screen 180, chin positioning motor, 314, joystick 320, and chin rest height adjustment switch 324. head rest 310, chin positioning motor 314, joystick 320, height adjustment switch of the chin rest 324, a self-refractor 120. Other embodiments have more or less components that communicate with the System Processor 402. In some embodiments, the IOCI 407 has buffers/drivers that simply provide appropriate drive voltage and current to the devices being controlled by the System Processor 402. In other embodiments, the IOCI 407 has additional processing capabilities that allow the IOCI 407 to take commands from the System Processor 402 and generate signals to one or more of the devices coupled to the IOCI 407.

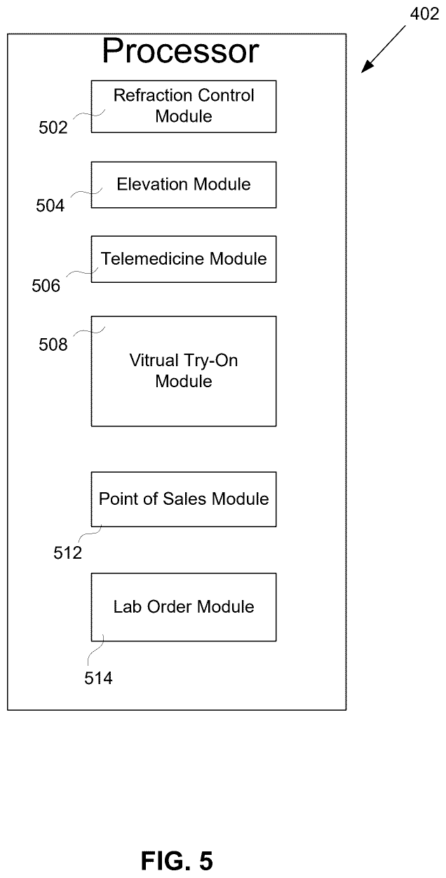

FIG. 5 is a more detailed illustration of the modules within the System Processor 402 in accordance with one embodiment of the station 100. In the embodiment of the System Processor 402 shown in FIG. 5, the System Processor 402 comprises a Refraction Control Module (RCM) 502, an Elevation Module 504, a Telemedicine Module 506, a Virtual Try-On Module (VTM) 508, a Point of Sale Module 512, and a Lab Order Module 514.

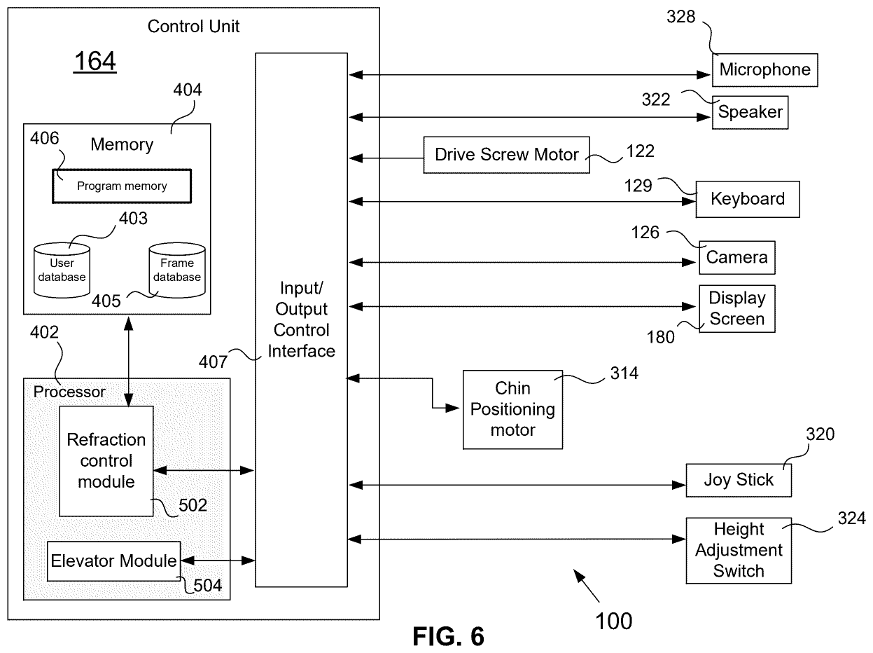

FIG. 6 is a simplified illustration showing the RCM 502, the IOCI 407, and some of the other components of the station 100. It should be noted that in order to simplify the drawing, some components of the station 100 shown in other figures, such as FIG. 4, have been omitted from this figure. However, it should be understood that these components would nonetheless be present in some embodiments of the station 100 of FIG. 6.

### Refraction Control Module

In some embodiments, the RCM 502 is executed by the System Processor 402 and controls the subjective self-refraction procedure. In some embodiments, the RCM 502 directly controls the components of the self-refractor 120 through the IOCI 407. In other such embodiments, the RCM 502 interfaces through the IOCI 407 with a central processor unit (CPU) 409 within the self-refractor 120 that controls the subjective self-refraction procedure in the manner disclosed in the '532 application and/or the '578 patent.

In some embodiments, the RCM 502 also provides control signals to the IOCI 407 which, then in turn, sends signals to the speaker 190 to provide voice instructions to the user. In some embodiments, the voice instructions guide the user through a step by step subjective self-refraction procedure. In some embodiments, the RCM 502 receives responses from the user via IOCI 407, which the user provides through user interface devices, such as the keyboard 129, touch screen 280, joystick 320, height adjustment switch 324, microphone 230, etc. The RCM 502 then processes the user responses. In some embodiments, the RCM 502 controls the subjective self-refraction procedure based on the user responses. In addition, the RCM 502 transmits further instructions to the user to instruct the user regarding the next steps in a subjective self-refraction procedure.

In some embodiments, the user enters information, such as the user's height information using the keyboard 129, or the touch screen 180. In some such embodiments, the IOCI 407 provides an interface between the keyboard 129 and the RCM 502. In other embodiments, the signals from the keyboard 129 are passed directly through the IOCI 407 to the Processor 402. In other embodiments, information received from keyboard 129 is stored in the user database 403, which can be accessed by the RCM 502. The data can be accessed and used to move the eye piece 308 to the desired height, at the eye level position of the user. Furthermore, in some embodiments, the RCM 502 uses the data to generate control signals sent through the IOCI 407 to the chin positioning motor 314 to adjust the height of the chinrest 312 so that when the user rests his chin on the chinrest 312, the user's eye is aligned with the eye piece 308 to allow an image of the user's eye to be captured during a subjective self-refraction procedure. In other embodiments, the user directly controls the height of the chinrest 312 using one of the motor height adjustment switch 324.

In some embodiments, the subjective self-refraction procedure comprises placing optical elements in the line of sight of the user and having the user look at a refraction viewing target. In some such embodiments, the optical elements are selected from: i) a spherical assembly comprising adjustable spherical optical power; and/or ii) an astigmatism assembly comprising adjustable astigmatism optical power. In some embodiments, in response to audible instructions from the station 100, the user responds by providing information indicating which one of the optical elements provides a clearer view of the refraction viewing target from among the choices presented to the user. In some embodiments, the information is in the form of instructions to the station 100.

The self-refractor 120 provides a 20 foot (or 6 meters) optical distance for user during the subjective self-refraction procedure. The self-refractor 120 presents eyecharts for the user to look at, including Snellen letters, tumbling E letters or a PSF refraction target, as disclosed in U.S. Pat. No. 8,867,801 to Shui Lai, incorporated herein by reference. A subjective self-refraction procedure is performed without an operator (i.e., technician or a doctor). In some embodiments, the steps of the subjective self-refraction procedure include one or more of the following: an auto-refraction including capturing wavefront data of the user's eye, visual acuity tests, Duochrome tests; and presenting at least two choices of optical powers, through which the user looks at a refraction target and the instrument asking the user to choose which one of the optical power presentations appears to be clearer to the user. In addition, it also performs procedures controlling over-minuses. In another embodiment, a short video is shown to the user on the display screen 180 prior to the subjective self-refraction procedure, thereby showing the user what to expect in the process. The more the user is familiar with the process, the less likely are the errors.

Subjective self-refraction procedures are thereby conducted in accordance with the disclosure provided by the '532 application. Alternatively, the self-refractor 120 follows the disclosure provided in the '578 patent.

### Telemedicine Module

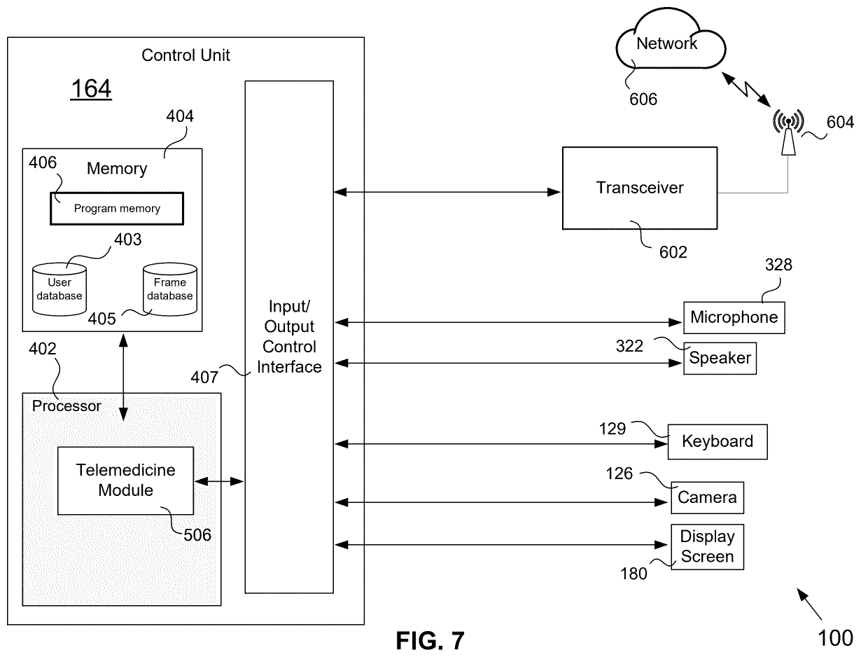

FIG. 7 is an illustration of parts of the station 100 showing the Telemedicine Module 506 and some of the associated components that interact with the Telemedicine Module 506. It should be noted that in order to simplify the drawing, some components of the station 100 shown in other figures, such as FIGS. 4 and 6, have been omitted from this figure. However, it should be understood that such omitted components are nonetheless present in some embodiments of station 100 shown in FIG. 7.

In some embodiments, the self-refraction procedure uses the Telemedicine Module 506 to provide refraction results from the subjective self-refraction procedure performed at the self-refractor 120 to a qualified professional, such as a refractionist. The refractionist is trained and certified to perform a subjective self-refraction procedure and review the refraction results. The Telemedicine Module 506 retrieves the refraction results from the user database 403. In some embodiments, the Telemedicine Module 506 is part of a telemedicine platform that includes a wireless network, the display screen 180, the camera 126, the microphone 328, the speaker 322. In addition, a remote speaker (not shown) and a remote display screen (not shown) that are located at the remote location provide a way to communicate with the refractionist at the remote location. Also provided are a transceiver 602, an antenna 604 and other communication hardware that enable the user to communicate with the refractionist at a remote location, including having a video conference call with the remote refractionist. In some embodiments, additional communication hardware comprises a network 606, such as a local area network, wide area network, Wireless Fidelity (WiFi) network, mobile telephone network or other such communication system. The network 606 allows communication, such as video conferencing, between the user and the remote refractionist. The Telemedicine Module 506 manages communication through the transceiver 602 and antenna 604 to establish a communication link with the network 606 through which the user can communicate with the refractionist, including receiving video and image files from the camera 126, audio from the microphone 328 and any data and graphics, data plots, provided by the self-refractor 120 and by the user through the keyboard 129, as well as displaying on the display screen 180 video and playing through the speaker 322 audio received through the network 606. In some embodiments, the transceiver is a modem. In some embodiments in which communication is performed in real time, the user and refractionist can speak directly to one another. This mode of operation is called a synchronous telemedicine service.

In some embodiments, a comprehensive eye exam is performed that includes pre-tests, a subjective self-refraction procedure, and a conference between the user and a qualified professional, such as a technician or doctor. The telemedicine module 506 allows the user to communicate with a doctor, as well as, or instead of a technician or other such qualified professional during the comprehensive exam. Details of the comprehensive eye exam are provided below.

### Comprehensive Eye Exams Using the Telemedicine Module

In some environments there are strict requirements regarding how a comprehensive eye exam is to be performed. In some such environments, a licensed doctor must perform subjective eye tests. Using the Telemedicine Module 506, it is possible for one doctor to oversee multiple locations comprising multiple optical offices and/or stations 100. Allowing the multiplexing of a doctor over several locations dramatically reduces the operating costs for performing such eye exams. In addition, such multiplexing makes it possible for one doctor to perform eye exams over a relatively large geographic region, thus providing relief from potential shortages of doctors in rural areas, and saving time and money that are otherwise necessary to recruit multiple doctors to work at multiple locations.

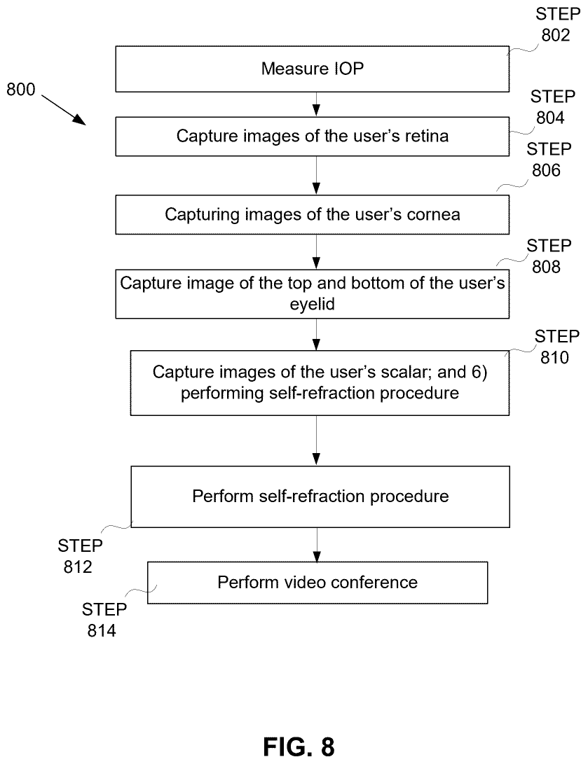

FIG. 8 illustrates a method 800 for performing eye tests that are required as part of a comprehensive eye exam in accordance with one embodiment of the disclosed method and apparatus.

In accordance with some embodiments of the presently disclosed method and apparatus, a comprehensive eye exam consists of three parts. In some embodiments, the first part includes performing pre-tests. The pre-tests comprise taking several measurements that in some embodiments are performed in response to a simple push of a button. In some embodiments, instruments that are used to perform the pre-tests have automatic focusing and automatic eye alignment capability. In some embodiments, the user sits behind the instrument and places his head on a head rest or a chin support. In some such embodiments, a technician moves the instrument to place the user's eye within the range of the instrument. When the user is properly positioned and ready to start the pre-tests, the user pushes a start button to activate the pre-tests. In other embodiments, the pre-tests can be remotely activated via the Telemedicine Module 506. In some embodiments, in response to the pre-tests being activated, the instrument performs movements to finely align the user's eye as part of the automatic eye alignment process. In addition, the auto-focus process is performed. Once the user's eye is aligned and focused, an image is captured.

In some embodiments, pre-test measurements are taken with the assistance of a remote ophthalmic technician communicating with the user through the Telemedicine Module 506. These pre-test measurements include: 1) measuring intraocular pressure (IOP) of the eyes of the user (STEP 802); 2) capturing images of the user's retina (STEP 804); 3) capturing images of the user's cornea (STEP 806); 4) capturing images of the top and bottom of the user's eyelid (STEP 808); and 5) capturing images of the user's scalar (STEP 810).

The IOP is measured with a unit, such as the tonometer mounted in the station 100. In some embodiments, the tonometer is triggered by the remotely located technician once the user is properly positioned. In other embodiments, sensors (not shown) within the station 100 determine whether the user is properly positioned and activate the tonometer. The IOP can be useful in determining signs of developing glaucoma.

In some embodiments, images of the user's retina are taken by a retinal imaging tool, such as a specially adapted Volk Pictor Plus mobile imaging tool. In some such embodiments, a remote technician or doctor triggers the retinal imaging tool to take the image. The images of the user's retina are then sent to the remote technician or doctor to be examined in order to determine the occurrence of certain eye conditions, including macular degeneration, diabetic retinopathy, etc. Analyzing the image of the retina also allows the progression of retinal diseases to be monitored over time.

In some embodiments, images of the cornea are taken by slit lamp biomicroscope (SLB). The SLB projects a thin slice of light onto the cornea. In accordance with some embodiments, the SLB has a camera that allows images to be recorded. The operation of the SLB may be controlled remotely by the remote technician or doctor through the Telemedicine Module 506. The images of the cornea allow a determination of the level of clarity of the cornea and the intraocular lenses of the eye to be made.

The top and bottom edges of the eyelid are examined to identify any signs of developing dry eye due to meibomian gland decay or blockage of oil lubricant secretion.

Images of the scalar of the user's eye are reviewed by the remote technician or doctor to determine any abnormality of the blood vessels.

In some embodiments, all of the images and data collected are sent for review to a remote technician and/or doctor through the Telemedicine Module 506. It should be noted that there is no particular order in which pre-tests are to be performed.

In accordance with the disclosed method and apparatus, the second part of the comprehensive includes performing a subjective self-refraction procedure (STEP 812). The subjective self-refraction procedure is performed as described above. Learning to refract with a phoropter requires a significant amount of training and practice. Due to the level of skill required to produce accurate and reliable subjective refraction results, attaining such results is exceptional rather than a rule among refractionist. By replacing the phoropter with a self-refractor 120, the amount of training required is reduced. In some embodiments no operator is present at the station 100. In other embodiments, an operator with minimal training, (i.e., sufficient training to observe the process not to conduct it) is provided at the station 100. Reducing the training required reduces the cost. In addition, having a less skilled person can extend the business hours during which such testing can be done. Accordingly, the disclosed method and apparatus results in a remote comprehensive eye exam that is more efficient, convenient and affordable.

In some of the embodiments, a technician present at the station 100 operates the self-refractor 120. In other embodiments, a technician operates the self-refractor 120 from a remote location. In some embodiments in which the subjective self-refraction procedure is activated by a technician from a remote location, the technician explains the subjective self-refraction procedure to the user. In some embodiments in which the self-refractor 120 has voice guidance, instructions for each step of the subjective self-refraction procedure is announced through the speaker 190, the operator of the self-refractor 120 is there to observe. By observing the subjective self-refraction procedure, the technician may assist if the user seems unable to follow the computer-generated instructions. Since the technician is not required to ask questions or provide input or adjustments during the subjective self-refraction procedure, the user performs the subjective self-refraction procedure by himself by following the instruction provided during subjective self-refraction procedure.

Accordingly, the subjective self-refraction procedure does not require the user to answer questions. In contrast, a typical subjective process using a phoropter requires the operator to ask multiple questions as different optical powers are presented to the user. In some embodiments of the disclosed method and apparatus, the user provides feedback by controlling an interface device, such as a joystick or a light box with multiple buttons. The feedback indicates preferences between optical presentations presented to the user. The users input from the joystick interface is analyzed by the self-refractor 120 to determine whether the optic is sphere, cylinder or the axis values, and how much to change the optical power from the last optics presented to the user. It should be noted that when such subjective refraction is performed by a technician that is not highly skilled, mistakes are common. Such mistakes lead to inaccurate refraction results. However, the subjective self-refraction procedure of the presently disclosed method and apparatus is not dependent on the operator. Accordingly, the subjective self-refraction procedure provides more accurate results and greater consistency from one patient to another. In some embodiments in which an operator is present, the operator is there merely to observe and remind the user to follow the instructions provided through the interface devices of the self-refractor 120, such as the voice commands provided through the speaker 190.

The presently disclosed subjective self-refraction procedure eliminates the requirement for a doctor to supervise a technician performing a preliminary refraction. In some embodiments, such supervision can be provided either locally or through the Telemedicine Module 506. In prior art procedures, having a technician perform a preliminary refraction minimizes the amount of time a doctor is required. In some embodiments, the technician or a doctor initiates the subjective self-refraction procedure. In some embodiments, the operator is supervised by the doctor. After initiating the procedure, the user continues the procedure by himself at the station 100. In such embodiments, the requirement that the doctor be at the same location as the operator is satisfied. Nonetheless, the user performs the subjective self-refraction procedure remote from the doctor who is connecting through Telemedicine Module 506. The amount of time saved is greater when either the technician or the doctor performs a subjective refraction; to initiate a self-refraction versus actually to perform a refraction.

Part three of the comprehensive eye exam is a video conference held between a qualified professional and the user (STEP 814). In some embodiments, the profession discusses if any disease conditions are developing or have developed. Instead of conducting an in-person interview, in some embodiments, the interview is conducted through the Telemedicine Module 506. Recommendations to see an eye specialist for the specific eye disease are provided to the user by the professional when warranted. In some embodiments, pre-testing is performed by an on-sight technician. In some embodiments, the on site professional is assisted by a licensed doctor through the Telemedicine Module 506.

In some embodiments, part three of the comprehensive eye exam may also include refinement or adjustment of the preliminary refraction results by the eye care professional. Using the Telemedicine Module 506, the eye care professional at the remote location may determine that some adjustments to the subjective self-refraction procedure are necessary. In some embodiments, if adjustments are required, the professional at the site of the station 100 communicates with the RCM 502 through the IOCI 407 via a software application that allows a computer (not shown) at the remote location to access the processor 402. In some embodiments, the remote computer controls the operation of the processor 402 as if the remote professional were at the station 100. In some embodiments, commercially available software applications, such as RemotePC, TeamViever, Splashtop, to name a few, can be used to remotely control the processor 402. Accordingly, the remote professional can make refinements to the subjective self-refraction procedure or perform a subjective eye test for the user through the station 100. When the remote person is satisfied with its adjustments, the refraction results are considered finalized by the remote person.

In some embodiments, the preliminary subjective self-refraction procedure takes place without the direct supervision of a technician or a refractionist. In some states, any operator or assistant of a subjective refraction device cannot legally conduct a subjective refraction unless it is at the same location as the licensed eye care professional. In the case of the subjective refraction performed by the user without an operator, the operator who has not operated the self-refractor could have been anyone of the technicians at the location with the licensed eye care professional. In some embodiments, the self-refraction is initiated by an operator at the doctor's location, or the user initiates the subjective self-refraction procedure. Therefore, the legal requirement is satisfied when the refraction is operated by the user itself, without an assistant.

### Virtual Try-on Module

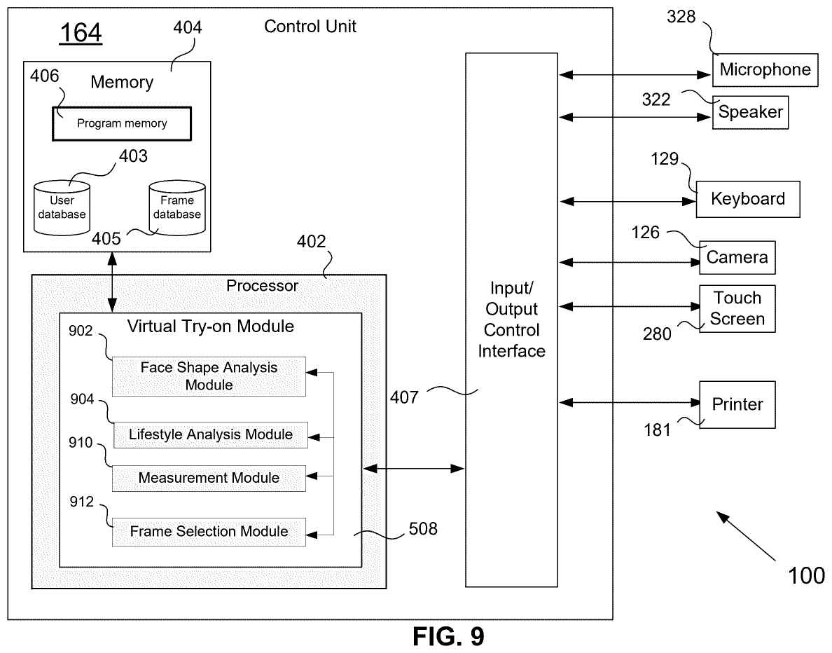

FIG. 9 is an illustration of part of the station 100, showing the VTM 508 and the associated components that interact with the VTM 508. As was the case regarding FIGS. 4, 6 and 7 above, in some embodiments, components shown in other figures, such as FIGS. 4, 6, and 7, are present in the station 100 shown in FIG. 9, but are omitted to simplify the figure.

The VTM 508 allows the user to see how physical frames may look on the user's face, prior to ordering the frames. The VTM 508 comprises a face shape analysis (FSA) Module 902, a Lifestyle Analysis Module 904, a Measurement Module 910 and a Frame Selection Module 912.

In some embodiments, the VTM 508 is coupled to the camera 126 through the IOCI 407. The camera 126 provides an image of the user's face to the VTM 508. In some such embodiments, the FSA module 902 and a Measurement Module 910 receive an image of the user's face and facial features from the camera 126.

### Face Shape Analysis Module

Initially, images from the camera 126 are calibrated to allow measurements of the user's face, pupil centers and segment height and to allow an accurate merging of the user's image with an image of a virtual frame. The FSA Module 902 performs a measurement calibration procedure to ensure an accurate determination of distance between points on the image.

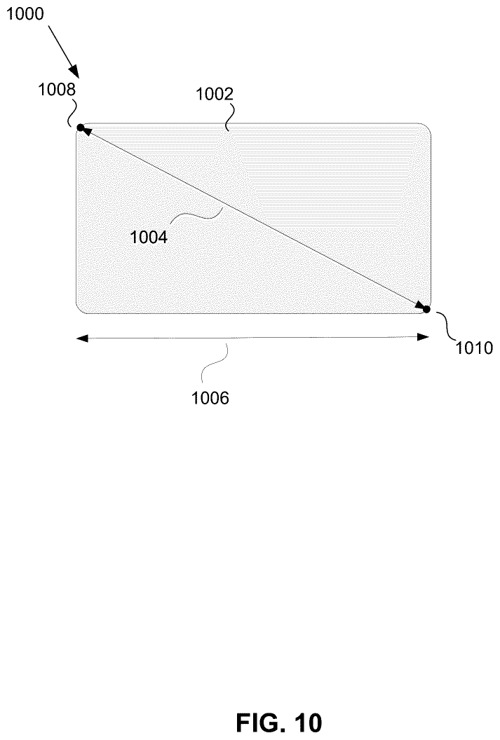

FIG. 10 is an illustration of a reference image 1000 used in the measurement calibration procedure to generate a calibration factor. In some embodiments, the reference image is a credit card. The measurement calibration procedure is performed by the FSA Module 902, generating an instruction to the camera 126 to take an image of a credit card 1002, which is placed at a predetermined distance and camera angle from the camera 126. Since all credit cards are made to a standard size (i.e., 3.375×2.125 inches), the distance across a diagonal 1004 of the card (or along one of edges 1006 of the card) is known. Therefore, with the card 1002 appropriately positioned, the number of pixels between the end points 1008, 1010 of a line 1004 across an image of the credit card 1002 is measured by the FSA Module 902. Knowing both the number of pixels and the actual distance between the first point 1008 and the second point 1010, the FSA Module 902 can determine a calibration factor to be used to determine the distance between any two points within any other image taken by the camera 126 at the same distance and camera angle based on the number of pixels between the points. Instead of the diagonal line of the credit card, one may use the end points of the magnetic strip, which present in most credit cards to find the calibration factor. In other embodiments, methods of converting the distance between pixels in an image to the distance between points in an image that are known to those skilled in the art are used.

In some embodiments, the distance at which the credit card (or other reference image) is placed to calibrate the image is selected such that when a user's face is placed at that same distance, the user's face will occupy ⅓ to ¼ of the screen height of the display monitor.

In some embodiments, when measuring facial features and frame dimensions, the measurement calibration procedure needs only to be performed once per user.

In some embodiments, a mark such as a line, a circle or an outline of a foot are painted on the floor in front of the station 100. The lateral distance of the mark is selected at appropriate distance from the camera 126. Once a measurement calibration procedure has been performed, with a user standing over a mark using a standard internal reference, such as a credit card shown in FIG. 10, the measurement calibration procedure need not be repeated for all other users standing over the marked location. Typically, a person's heel is aligned with their head when they are standing. The position of the face is relatively fixed relative to the head, thereby positioning the user's heel at the designated marked location on the floor, the position of the user's face is also fixed. The calibration factor obtained from the measurement calibration procedure may be used for all subsequent users if they stands with their heel over the mark.

In some embodiments, the FSA Module 902 sends a signal to the IOCI 407 that is in turn sent to camera 126 to take a picture of the user's face. In some embodiments, the signal generated by the FSA Module 902 is triggered by a user input to the FSA Module 902 provided through a user interface device, such as by pressing a designated area on the touch screen 180 or 280. In other embodiments, the user input is a camera clicker that directly triggers the camera 126 to initiate a face shape capture.

The image taken by the camera 126 is sent back through the IOCI 407 to the FSA Module 902 within the Processor 402. The FSA Module 902 uses the image to determine the shape of the user's face. In some embodiments, the shape of the user's face is determined by comparing the image taken by the camera 126 with reference images to determine whether the user's face is “round”, “square”, oval, triangular, diamond, etc. It should be understood that other embodiments may use a different set of characterizations (such as “long”, “compact”, etc.) to classify the user's face. In addition, in some embodiments, the relative size of the user's face is determined. The set of user facial characterizations (i.e., the shape and relative size) are made available to the Frame Selection Module 912. In some embodiments, the set of user facial characterizations is stored in the user database 403, from which the Frame Selection Module 912 can read. In other embodiments, the user's facial characterizations are coupled directly from the FSA Module 902 to the Frame Selection Module 912.

### Lifestyle Analysis Module

As will be discussed in further detail below, when the user begins the process of receiving services from the station 100, the user provides information to the station 100 through the user interface devices. In some embodiments, this information includes information about the user that might affect the user's decision as to which frame might be most satisfying to the user. The Lifestyle Analysis Module 904 includes a set of rules governing the lifestyle types with colors, frame shape, frame material, frame size, crystal enhancements, brink levels, tints, mirror coatings, etc. The rules are applied to the user's answers to a lifestyle questionnaire to create a set of frame characteristics best suited for the user.

That is, when a user buys eyeglasses, there are many factors that can affect the user's selection of a frame. In addition to matching the frame to the user's face, one's lifestyle and fashion preferences can affect the choice of the frame's color, material (i.e., plastic versus metal types, semi-metal, etc.), whether the frame is rimless or semi-rimless, etc. For example, a conservative person may prefer less flashy frames, in a darker color, square-shaped frames. A more flamboyant person might prefer a flashy star-shaped red frame. When someone wants people to notice them, and to stand out in a crowd, their choice may be an oversized frame, with half-tinted lenses, decorated with crystals, and metal trimmings on the frame or on the temples. Furthermore, some people prefer a frame designed by a well-known designer. Outdoor people, like fishermen, golfers or cyclists, might prefer a sporty-looking frame made with strong and thicker construction. Additionally, some consider mirror coating in bright colors, or rainbow combination colors, to be features that enhance the sporty nature of a frame.

In addition, for some sportsmen, technical solutions that provide enhanced visibility, such as by reducing glare, enhancing the scenery by deepening nature's green color, or features that assist a golfer with seeking golf balls on a golf course. Other users may prefer lenses coated with color filters that enhance relaxation and reduce stress, anti-reflective coatings to cut out glare, etc. Still further, certain frame shapes are used to enhance personality. For example, some users may like aviator-shaped frames. “Cat eye” shape frames can emphasize intriguing and mysterious aspects in a lady.

In some embodiments, a lifestyle questionnaire is given to a user to determine the lifestyle of the user. In some such embodiments, the questionnaire asks:
(1) what the user is currently using eyeglasses for;
(2) what is the intended use for new glasses (i.e., the daily activities of the user);
   (a) what percentage of time will the glasses be used for work, leisure or hobby,
   (b) what percentage of time will the glasses be used to see objects at a distance,
   (c) what percentage of time will the glasses be used to see objects that are close (i.e., reading, typing, etc.),
   (d) what percentage of time do you need to see intermediate distance that is slightly farther than reading up close (i.e., viewing a computer screen),
(3) is there a specific impression the user would like to make when wearing the glasses;
   (a) Stand out in a crowd,
   (b) fun,
   (c) to create hype,
   (d) I want to be left alone, conservative,
   (e) a combination of (a) through (d)
(4) the nature of the user's work and when the glasses are likely to be worn;
   (a) in the office (i.e., computer work),
   (b) Outdoors,
   (c) while operating certain types of equipment (i.e., driving a truck, airplane, performing construction), etc.
(5) Which best fits the user's budget:
   (a) practical economic, traditional. everyday use,
   (b) premium quality (elegant and refined, customized),
   (c) Exclusive, Heightened Luxury.

If the glasses are used mostly at near or intermediate distance, follow-up questions may be asked, such as: i), the user's age, and ii), does the user need reading glasses? This information can be used for prescribing PAL (progressive addition lenses) for the user.

In some embodiments, users are presented with lens options. In some such embodiments, the lens options offered are based on one of more of the following factors:
(a) age of the user,
(b) single vision or progressive lenses,
(c) Lens Materials.

The lifestyle information is then compiled and processed. The results are made available to the Frame Selection Module 912 to be used to reduce the number of frames presented to the user based on the type of frame shape, frame material, frame color, level of blinks, etc. In some embodiments, the lifestyle information is stored in the user database 403 where it can be read by the Frame Selection Module 912.

In some embodiments, each of the frames in the frame inventory may include lifestyle characteristics such as color, frame shape, frame material, coatings, etc., that specify for which lifestyle the frame is best suited. In addition, frame information, such as the temple length, bridge width (DBL), and lens size (A) is associated with each frame.

### Frame Selection Module

The Frame Selection Module 912 receives the set of user facial characterizations provided by the FSA Module 902 and the lifestyle information provided by the Lifestyle Analysis Module 904. In some embodiments in which these characteristics and information are stored in the user database 403, the Frame Selection Module reads the user facial characterizations and information from the user database 403. In other embodiments, the user's facial characterizations and information are coupled to the Frame Selection Module 912 from both the FSA Module 902 and the Lifestyle Analysis Module 904.

In some embodiments, the Frame Selection Module 912 uses the experience of fashion experts to determine what frames should be matched to particular user characteristics and lifestyle information. In some such embodiments, an artificial intelligence engine is trained to match the characteristics and information to the frames that are most likely to please the user. In some embodiments, the Frame Selection Module 912 uses rules derived from the experts to associate face shapes with particular categories of frames. Accordingly, the rules guide the selection of frames. For example, frames with a round shape are considered to be best suited to square faces. Square-shaped frames are considered to be best suited to round faces. Applying the rules, when selecting a frame, results in the user selecting a frame that complements the user's face. In some embodiments, based on the rules, each frame is assigned to certain face shapes. The frames in the associated category form a list of frames that are available in the frame database 405 that are recommended for the particular user based on the information received from the Lifestyle Analysis Module 904 and the user's facial characterizations determined by the FSA Module 902. Each of the recommended frames fits and complements the user's face.

Since the Frame Selection Module 912 selects a subset of frames from the frame inventory based on rules to ensure that a recommended frame fits and complements the user's face, the number of frames presented to the user is limited. In some embodiments, no more than 20 frames are recommended to the user. In other embodiments, the number is greater than 20, but in any case, the number of frames presented is substantially less than the total number of frames in the inventory. In some embodiments, there are several hundred virtual frames in the frame database 405. Hence, the Frame Selection Module 912 substantially reduces the time and effort it would otherwise take a user to select an attractive frame by allowing users to select a frame from a small subset of available virtual frames, rather than looking through all of the frames in the inventory.

A composite image is generated by the FSA Module 902 by superimposing the image of the virtual frame on the image of the user captured by the camera 126. The FSA Module 902 is coupled through the IOCI to the display monitor 180. The FSA Module 902 provides signals to the display monitor 180 to display the composite image on the screen of display monitor 180. In other embodiments, the display monitor is the touchscreen monitor 280. Displaying the composite image allows the user to see himself on the display screen 180 virtually wearing the virtual frame (i.e., virtually try on the frame depicted by the virtual frame). In some embodiments, the composite image is a real-time virtual 3D image. In some embodiments, the FSA Module 902 maintains the proper location of the virtual frame on the user's head as the user's head moves. If the selected frame appears too large or too small for the user's face, the user can select a different frame, and a new composite image with the newly selected frame would be generated for the user to view.

In some embodiments, the camera 126 and the display screen 180 are mounted in a manner that allows their position to be adjusted. In some such embodiments, the display screen 180 and camera 126 are mounted on a moving platform (not shown), thus allowing the height of both to be adjusted. In some embodiments, the position of the moving platform is controlled by the Elevation Module 504. Adjusting the height of the camera 126 allows the camera 126 to point directly at the face of the user on a horizontal plane with the user's eye when virtually trying on a frame, thereby capturing a frontal image of the user. Placing the camera 126 on the same horizontal plane as the user's eye reduces the potential for image distortion, which can create inaccuracy in measurements taken from the image. In other embodiments, other available virtual try-on technologies are used.

In some embodiments, an inventory of virtual frames is loaded into the frame database 405. The virtual frames are generated by capturing images from an actual frame at various viewing angles, such as frontal, 90 degrees, 45 degrees and a top-down view. Accordingly, the generated virtual frame depicts an actual frame that is available to the user to purchase.

In some embodiments, the virtual frames are organized in the frame database 405 by categories, based on frame characteristics provided by the frame manufacturer. These characteristics include temple length, the bridge length (DBL), the lens width (A), the color, frame shape, frame material, and in addition, importantly, the type of face shapes for which this frame is suitable.

### Measurement Module

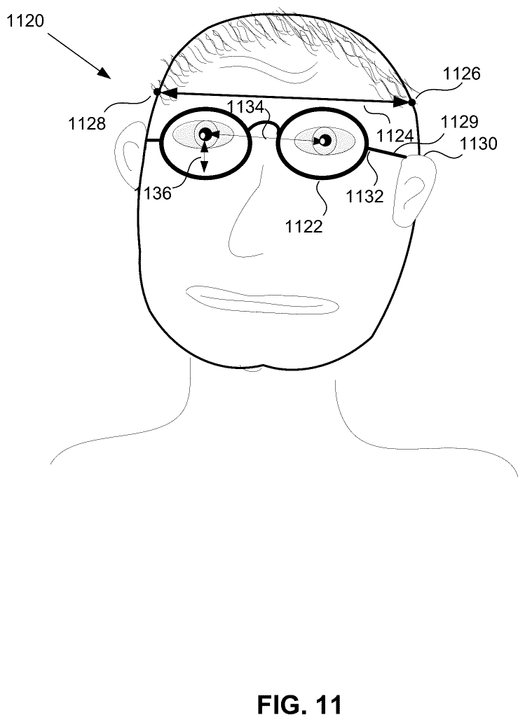

FIG. 11 is an illustration of a composite image 1120. The Measurement Module 910 uses the composite image 1120 of the user virtually wearing the virtual frame 1122 generated by the FSA Module 902 to determine the user's pupil distance 1134 and the user's segment height 1136 (i.e., the distance from the bottom edge of the user's pupil to the inside bottom edge of the frame) to allow lenses to be made. Once the user selects the frame, the Frame Selection Module 912 generates a virtual frame image for the selected frame, and the Measurement Module 910 measures the virtual frame of the composite image. Frame parameters are provided by frame manufacturer. The parameters are well known in the industry as the frame's A, B, ED, DBL numbers, where A is lens opening width at widest point, B is the height of the lens opening, ED is the effective diameter (measured as twice the distance from the geometric center of the lens to the farthest edge of the lens shape) and DBL is the temple length, the bridge width. Using the value of the frame parameters, the size of the virtual frame measured in the composite image using the calibration factor, is adjusted to match physical frame size as provided in the frame parameters provided by the manufacturer. The calibration factor is used in Measurement Module 910 to perform measurements on the virtual frame parameters and adjusts the dimensions of the virtual frame to its actual physical values (provided by the frame manufacturer). Thereby, the composite image of user virtually wearing the adjusted virtual frame would appear the same size as the actual physical frame is on the user's face. The result is a realistic image of the physical frame on the user's face.

Frame parameters provided by the manufacturer are maintained within station 100 in the frame database 405. Alternatively, such information can be maintained in a remote database that can be accessed by the station 100.

In some embodiments, the Measurement Module 910 generates instructions communicated to the user through the user interface devices, such as the speaker 322 or display screen 180, to request that the user turn his head 90 degrees to show his ear. The Measurement Module 910 communicates a command to the camera 126 through the IOCI 407 to capture an image once the user has turned his head. In some embodiments, the trigger for generating the signal to the camera 126 is provided by the user activating a user interface device. Accordingly, an image of the side of the user's head (including the user's ear) will be captured and provided to the Measurement Module 910.

The Measurement Module 910 measures the pixel distance 724 between two points 726, 728 of the image 720 of the user to determine the user's head size. In addition, the Measurement Module 910 measures the pixel distance 729 from the top of the ear 730 to the corner of the frame 732 where the eye glass temple connects to the front at the frame's hinge. This measurement 729 is then used to determine the point along the eye glass temple where a slight bend at the temple can be appropriately placed. Properly positioning the bend allows the temple to rest on the top of the ear (i.e., on the soft bone of the ear). A proper temple adjustment would allow the frame to sit properly over the user's ear. Accordingly, the measured values are then applied to properly adjust the frame temple. This results in the frame securely resting on the user's face, and helps to prevent the frame from slipping and sliding off the face. Pre-adjusting the frame at a central fabrication center (preferably at a quality control department) results in the frame fitting the user better, better customer satisfaction, and fewer customers seeking post-sale support.

### Point-of-Sale Module

The station 100 incorporates a Point-of-Sale Module 512. It comprises a payment facility, for example, having electronic payment hardware. An example of such hardware is a unit manufactured by PAX, model IM20. The Point of Sale Module 512, includes a communication network, connecting to the frame database 405, and lens material and add-on options database, collecting the price of all items from these databases, in the purchase order, and showing that amount for a user to make payment. Users swipe a credit card, tap a payment screen using the chip on a card, or pay using apps on a smartphone to pay for a purchase. After the payment went through, debited to the credit card, etc., the Point of Sale Module 512 sends a lab order to the fabrication labs to fabricate the lenses and insert the lenses in a frame chosen by the user during the virtual try on process at the self-service optical station.

Upon completing the point-of-sale transaction, a receipt is printed at the printer 181 and provided to the user.

### Lab Order Module

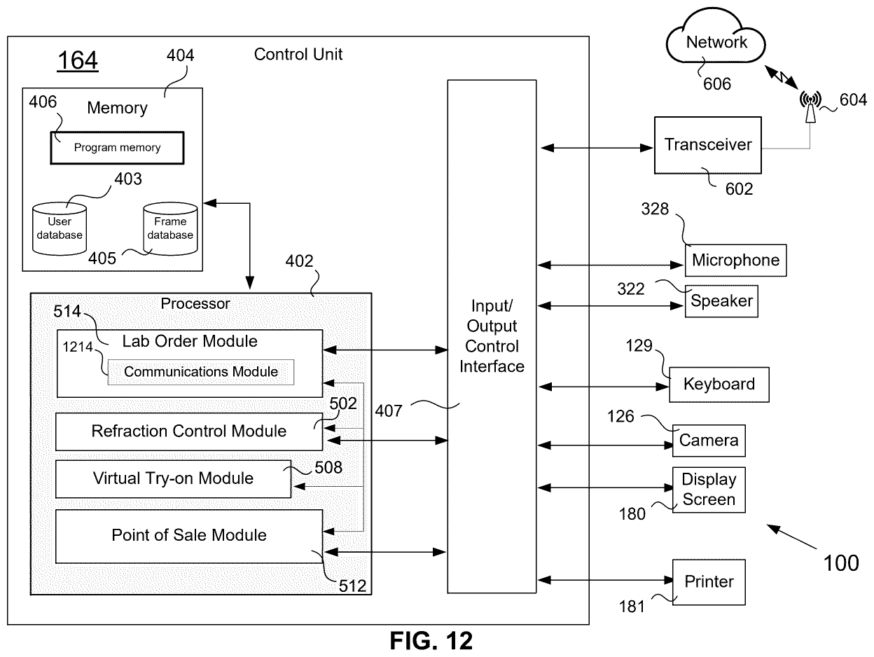

FIG. 12 is an illustration of an embodiment of the control unit 164 having a Lab Order Module 514 and some of the elements of some embodiments of the disclosed station 100 related to the Lab Order Module 514. The Lab Order Module 514 in the station comprises a Communication Module 1214. The Communication Module 1214 is coupled to the RCM 502 to collect refraction results. In addition, the Communication Module 1214 is coupled to VTM 508 to collect the identity and the frame parameters of the frame selected by the user, the pupil distance of the user, and segment height of the user. Communication Module 1214 is also coupled to the Point of Sale Module 512 to receive information indicating whether the correct payment was collected from the user for the purchase of the eyeglasses and the exam. Once all the above information has been collected, the Lab Order Module 514 sends the collected values to a fabrication laboratory to make the lenses and assemble them in the selected frame. The fabrication facility has a frame inventory of physical frames corresponding to the virtual frames in the frame inventory maintained in the frame database 405 of the station 100. The fabrication laboratory responds by completing the lab order. An inventory personnel pick out the physical frame selected by the user, manufactures the lenses as ordered by the user, inserts the lenses into the frame. After eyeglass fabrication is completed, a pair of eyeglasses are cleaned and properly packaged at the fabrication facility. The completed pair of eyeglasses are sent to the user using the address provided by the user during the user check-in process. Alternatively, the user may be contacted, such as by text or e-message, that their eyeglasses are ready for pickup at a specified location. In some embodiments, the location provides locked boxes secured by key codes provided to the user.

### Purchasing Eyeglasses Without Third-Party Assistance

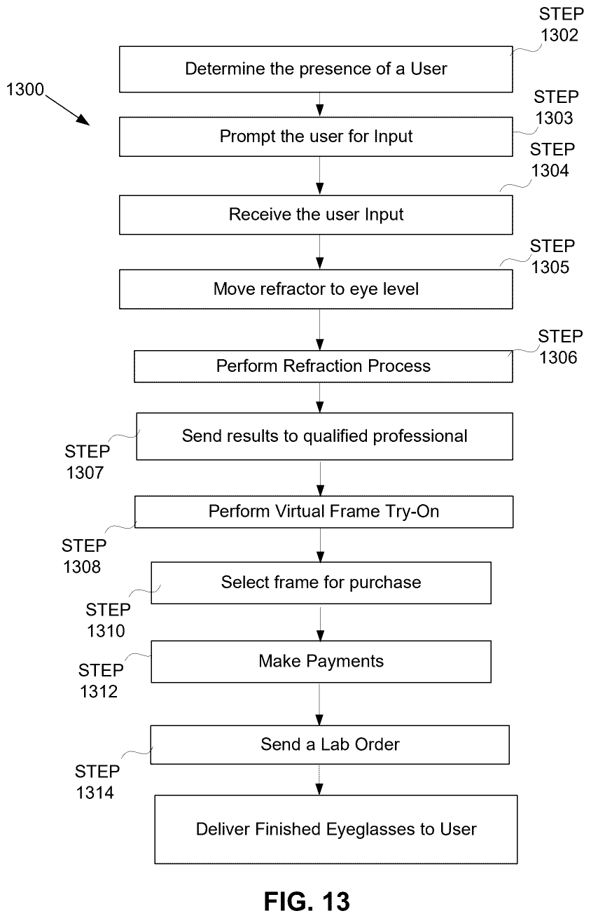

FIG. 13 is a flowchart of an embodiment of a method for performing a self-service optical transactions 1300 in accordance with the disclosed method and apparatus. In some embodiments, a user uses the station 100 to perform services including eye tests and purchasing eyeglasses, without third-party assistance. In some embodiments, a user walks up to the station and the presence of the user is determined by a sensor (STEP 1302). In other embodiments, the user initiates the services by providing a trigger to the station 100 through a user interface device, such as the touch screen 280.

Upon determining that a potential user is present, station 100 uses the speaker 190 to prompt the user to provide initial information about the user (STEP 1303) through voice instruction from the station 100. The user provides information received by the station 100 (STEP 1304) as part of a check-in procedure. In some embodiments, the user provides his/her email, phone number, and address to which completed pair of eyeglasses are to be sent. User provides his/her height information. Once the user information has been received, the Elevation Module moves the self-refractor 120 to the eye level of the user (STEP 1305). In some embodiments, the self-refractor 120 is stationary, and the user adjusts himself/herself to align his/her eye with the optical entrance of the self-refractor 120. The user may stand on a stool if the user is too short or the user may spread his/her the legs if the user is too tall, placing his/her eye against the eyepiece 308.

A computerized voice from the station 100 provides audible instructions to the user to perform a subjective self-refraction procedure (STEP 1306). In some embodiments, the user initiates the subjective self-refraction procedure through inputs to the user interface devices in response to the audible instructions. In other embodiments, station 100 initiates the subjective self-refraction procedure automatically after receiving all of the requested user input. As described in earlier sections, In some embodiments, the subjective self-refraction procedure is performed under the control of the RCM 502 while in other embodiments, the RCM 502 communicates with the CPU 409 within the self-refractor 120 to perform the subjective self-refraction procedure. In some embodiments, users also perform a list of other eye tests including an auto-refraction procedure comprising capturing of wavefront data of the user's eye, visual acuity tests, duochrome tests, controlling over-minus, by itself, without assistance of a technician or a doctor.

After the subjective self-refraction procedure has been completed, the station 100 sends the results to a qualified professional (STEP 1307) through Telemedicine Module 506. The professional reviews the results of the subjective self-refraction procedure and validates the results. Next, the computer voice provided through the speaker 190 guides the user to virtually try on frames using the VTM 508 (STEP 1308).

As part of the virtual try-on procedure, the station 100 instructs the user through the speaker 190 to stand over a designated mark on the floor. The mark on the floor allows the station 100 to perform the measurement calibration procedure detailed above. The camera 126 takes a portrait of the user. Using the portrait, the FSA Module 902 applies information provided by the Lifestyle Analysis Module 904 within the VTM 508 determine a subset of frames that are appropriate to present to the user. In addition, the FSA Module 902 combines each presented frame with the portrait of the user to show the user virtually wearing the virtual frame in 3D.

After reviewing multiple virtual frames, the user reduces the number of frames by selecting frames to try on from the set of frames offered to the user as part of a virtual try-on procedure. After trying on one or more of the virtual frames, the user selects one of the frames (STEP 1310) for purchase. User proceeds to select the lens material and any add-ons to the lenses to make the prescription lenses using the prescription from the self-refractor measurements. The user then pays for the frames and the lenses through the Point-of-Sale Module 512 within the processor 402. The payments are received by through the System Processor 402 (STEP 1312). Upon receiving payment, the station 100 sends a lab order to a fabrication laboratory (STEP 1314). Once the lab order is sent, the procedure is complete and the finished eyeglasses are delivered to the user by the fabrication laboratory and finished eyeglasses arrive at the user's home (STEP 1316).

From the user's point of view, he/she performed all necessary steps by following the instructions provided at the station resulting in a finished pair of eyeglasses appearing at the user's door step. The entire process is performed as self-service process, all without third party assistance.

In some embodiments, assistance may be provided by personnel present at the station 100. Such personnel may be there to provide information to the user and explain how the subjective self-refraction procedure proceeds, so the user will know what to expect. Such assistance helps to reduce user confusion or user errors during the subjective self-refraction procedure. The personnel in these cases, is not conducting the subjective self-refraction procedure, but is there to observe. Such personnel may intercede when the user appears to stray from or misunderstand certain steps of the subjective self-refraction procedure. In other embodiment, the assisting personnel is a refractionist at location remote from the station 100. A video conference is established between the user and the assisting personnel during the subjective self-refraction procedure. The user can see and hear the assisting personnel through the video conference established through the Telemedicine module 506. In some embodiments, the assisting personnel remotely controls the subjective self-refraction procedure.

It should be noted that in some embodiments of the present invention, there is no need to provide a chair for the user. Accordingly, the expense of the chair and the need to clean/sterilize the chair is eliminated. In embodiments that use a chair, cleaning is required after each use. This requires an attendant to visit each station 100, increasing the operating cost. Costs and complexity can be reduced for stations 100 that are in operation and open to the public 24 hours a day, 7 days a week, by having less elements to clean or sterilize.

### Refraction Procedure

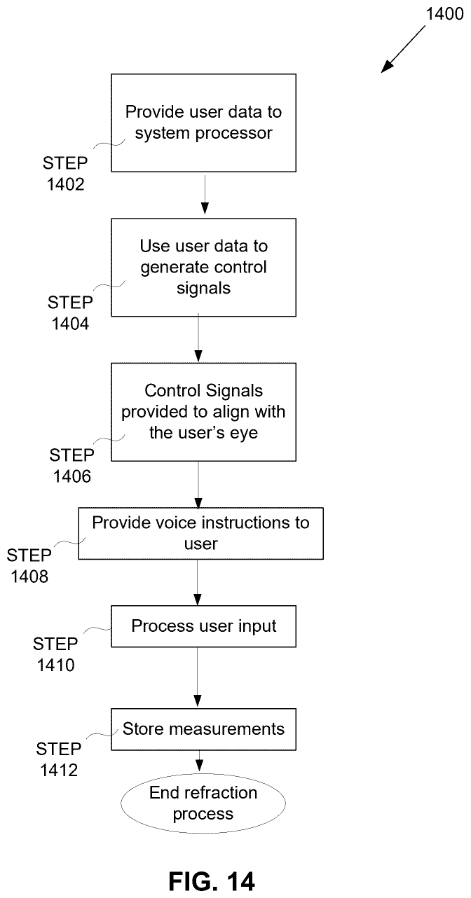

FIG. 14 is a flowchart of one embodiment of the subjective self-refraction procedure 1400. In some embodiments, user data stored in the database 403 is provided to the System Processor 402 (STEP 1402). The user input data allows the System Processor 402 to generate control signals that control various functions of station 100 (STEP 1404). For example, after receiving and storing the user's height in the System Processor 402 provide control signals to the Elevation Module 504 to drive the drive screw motor 122, moves the platform 121 with the self-refractor 120 to a height that places the eye piece 308 at the user's eye level (STEP 1406).

Once the self-refractor 120 has moved into position, the user is instructed by a computerized voice generated by a program within the self-refractor 120. In some embodiments, guidance is output from the speaker 190 to issue voice instructions to the user to place his eye against the eyepiece 308 of the self-refractor 120, which is the optical entrance of the subjective self-refractor 120. The user looks inside the self-refractor 120. Voice instructions are provided to the user to start a subjective self-refraction procedure (STEP 1408). The procedure comprises some of the eye tests, including visual acuity tests, auto refraction comprising capturing wavefront data of the user's eye, subjective eye tests, wherein the user decides while looking at the refraction target, which choice of the presented optical powers appears clear, duochrome tests and tests controlling over minus.

In some embodiments, before the subjective self-refraction procedure begins, training instructions are provided in the form of a video or a slide presentation explaining the subjective self-refraction procedure to prepare the user to provide proper responses in various stages of the subjective self-refraction procedure. In some embodiments, station 100 has a voice recognition program, allowing it to interpret the speech or the voice responses by the user.

In response to the instructions provided to the user, the System Processor 402 receives input from the user (STEP 1410). The user input is then processed by the system processor (STEP 1412), to adjust optics in the self-refractor 120.

After a subjective self-refraction procedure is completed, the results are stored (STEP 1414) and patient in-take forms are sent with the results to a qualified refractionist for approval through Telemedicine Module 506. Upon the approval by the qualified refractionist, a prescription certificate is issued by a supervisor ophthalmologist or an optometrist to the user.

In some embodiments, the subjective self-refraction procedure and associated measurements are made using the refraction device and the associated method disclosed in the pending '532 application. Alternatively, the subjective self-refraction procedure and associated measurements are made using the refraction device and associated method disclosed in the '578 patent. While the disclosed method and apparatus is described as using the devices disclosed in the '532 patent and the '578 patent, it should be understood that other self-refraction devices may be used instead.

### Economic Benefits of the Self-Service Optical Stations

The following economic benefits are derived from some of the features of some embodiments but should not be used to define whether a particular feature is present or not. That is, while these benefits flow from the presence of one or more of the features of the disclosed method and apparatus, particular benefits may not be attached in all embodiments of the disclosed method and apparatus. Accordingly, the features recited in the claims attached hereto should determine the invention and the presence or absence of one or more of the following benefits should not define the claimed invention, but rather are disclosed herein merely as an example of some benefits that might be derived from the disclosed method and apparatus.

### Constructing the Self-Sufficient Optical Station in a Compact Footprint

The self-service optical station is constructed as a free-standing unit in a form factor of a kiosk. The station provides many of the essential optical services of a brick-and-mortar optical store, including performing eye tests and selling eyeglasses. However, the stations operate without attendants. The technologies housed in this compact structure replaces manual labor with computer-guided measurements and services, thereby substantially lowers the operating costs of performing optical transactions.

From FIG. 1, the entire station is constructed with a small footprint, in the form of a compact kiosk. It integrates several key technologies at the station. Referring to FIG. 1, in this example, the footprint is about 4 ft wide by 4 ft in depth (16 square feet) with a form factor of a small-sized kiosk. The advantage of having a small footprint is a lower rent. The rent of a 16 square foot kiosk is substantially less than that of a 2,000 square foot space of a typical brick and mortar optical store. The small size also makes it easier to find rental spaces for its hosting locations, in airports, college campuses, shopping malls, department stores, convention centers, grocery stores, pharmacy stores, among others.

### Lower Costs per Station Offering More Installed Locations, and More Convenience for Users

The startup costs of one self-service optical station are substantially less than those of a brick-and-mortar store. Adding the costs for tenant improvements, frame display, physical frame inventory in a typical optical store, a self-service optical station costs about one-tenth of those of a typical optical store. For the price of one traditional optical store, there can be 10 self-service optical stations, due to the cost advantages of using such stations. The result is more service locations, more convenient for users seeking optical services. Since the stations do not require any operator or attendant, the stations can be open 24 hours a day and 7 days a week.

### Self-Refraction Saving Costs and Time

The subjective self-refraction procedure includes a host of tests, such as auto-refraction and subjective self-refraction procedures. The entire subjective self-refraction procedure is done without an operator, thus eliminating costs associated with recruiting, paying and managing an operator. In some embodiments, computer software stored in the memory unit 404 coupled to the processor 402 communicates instructions through the IOCI 407 to the CPU 409 within the self-refractor 120 to execute the subjective self-refraction procedure step by step. The computer software is a cumulation of refraction expertise and bult-in artificial intelligence (AI) as disclosed in the '532 application noted above. Software within the memory unit 404 is capable of reacting to each of the user's responses. This enables users to perform a subjective self-refraction procedure. Furthermore, users do not need to make a doctor's appointment, eliminating the associated cost and time spent in a doctor's waiting room. The cost of paying a doctor to work at an optical store constitutes a substantial portion of its operating costs. Therefore, not needing a doctor at a refraction station in this business method constitutes a substantial saving. Alternatively, the RCM 502 within the processor 402 directly controls the self-refractor 120. That is, rather than having a CPU 409 within the self-refractor 120, the RCM 502 performs those functions that would otherwise be performed by the CPU 409 within the self-refractor 120.

### Computerized Measurements Replacing Opticians Onsite

Traditionally, an optician performs manual measurements of the PD and Segment Height. With the presently disclosed method and apparatus, computerized measurements are performed through the Measurement Module, providing higher accuracy than a manual measurement performed by an optician. The station eliminates the need, and the costs associated with hiring an optician. This computerized self-measurement, of the PD and segment height enables cost saving.

### Telemedicine Platform Time-Sharing One Refractionist

As mentioned earlier, the refraction results are transmitted through the telemedicine platform through the Telemedicine Module 506 to a qualified refractionist at a remote location. One such refractionist can oversee multiple refraction stations, instead of one refractionist or one doctor at each brick-and-mortar office. In accordance with the disclosed method and apparatus, one refractionist can service up to 15 to 20 locations. The cost of refractionist per station is lowered by 1/15^{th} or 1/20^{th} compared to that of a brick-and-mortar operation.

### Virtual Frame Try-on Offering More Frame Selections

The virtual try-on process through the VTM 508 enables users to access many more frame choices, from the frame inventory database 405, in comparison to the limited number of frames in an optical store. The frame selection in the inventory is only limited by the memory space, in which thousands or more frame images are stored on a terabyte memory chip, versus the limited display space in a brick-and-mortar store. Stocking physical frames are substantially more costly than stocking virtual frames on a memory chip. The frame database 405 can easily offer 10 times more frame selections than in a typical optical store.

By providing the user with the ability to virtually try on frames, the VTM 508 eliminates the need to stock physical inventory of frames at each station 100 and to clean the physical frames after each use. Eliminating the physical frame inventory at each station is a huge cost savings. Virtual frames do not have the risk of transmitting viruses. The FSA 902 is coupled to the Lifestyle Analysis Module 904 and enables a more comprehensive frame selection process by searching through the entire frame inventory based on the user's face shape and lifestyle choices. The FSA 902 narrows the results to a handful of best fitted frames matching the face shape and lifestyle choices of the user, thus saving time and reducing the confusion that would otherwise result from having to look through hundreds of frames to find a matching pair.

The self-service optical stations cuts operation costs, requires less startup costs, and less people costs and less management costs. The self-service optical stations offer more convenience by offering more convenient locations, and improving access to buy eyeglasses for users.

It should be understood that the order in which steps of the disclosed method shown in FIGS. 13, 14 and 15 are performed is an example and that these steps can be performed in any order. As such, these are examples only and should not be construed as limitations of the disclosure in the specification.

Although the disclosed method and apparatus is described above in terms of various examples of embodiments and implementations, it is understood that the particular features, aspects and functionality described in one or more of the individual embodiments are not limited in their applicability to the particular embodiment with which they are described. Thus, the breadth and scope of the claimed invention should not be limited by any of the examples provided in describing the above disclosed embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide examples of instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that are available or known now or at any time in the future. Likewise, where this document refers to technologies that would be apparent or known to one of ordinary skill in the art, such technologies encompass those apparent or known to the skilled artisan now or at any time in the future.

A group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the disclosed method and apparatus may be described or claimed in the singular, the plural is contemplated to be within the scope thereof unless limitation to the singular is explicitly stated.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described with the aid of block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A self-service optical station, comprising:
a) a structure having:
i) an enclosure; and
ii) a pedestal supporting the enclosure;
b) a self-refractor housed within the enclosure, the self-refractor configured to perform a subjective self-refraction procedure;
c) an eyepiece optically coupled to the self-refractor providing an optical entrance for a user's eye; and
d) an Elevation Module coupled to the self-refractor, the Elevation Module configured to move the eye piece of the refractor to the eye level of the user.

2. The self-service optical station of claim 1, comprising:
a) a speaker; and
b) a control unit comprising:
i) a processor comprising a Refraction Control Module (RCM) coupled to the self-refractor and to the speaker for providing audible instructions to the user on how to perform the subjective self-refraction procedure; and
ii) at least one user interface device coupled to the RCM for receiving information from the user directed to the RCM in response to the audible instructions on how to perform the subjective self-refraction procedure.

3. The self-service optical station of claim 2, wherein the at least one user interface device is further configured to receive information from the user regarding lifestyle choices, the processor further comprising:
a) a monitor: and
b) a Virtual Try-on Module (VTM) comprising;
i) a Face Shape Analyzer (FSA) Module configured to analyze the image of the user's face;
ii) a Lifestyle Analysis Module coupled to the FSA Module, the Lifestyle Analysis Module configured to receive input from user interface devices regarding user lifestyle choices; and
iii) a Frame Selection Module coupled to the FSA Module, the Lifestyle Analysis Module and the monitor, the Frame Selection Module configured to:
A) select a subset of frames from among a frame inventory based on information received from the FSA Module regarding the user's face and the Lifestyle Analysis Module regarding the user's lifestyle choices;
B) combine the image of the selected frames with the image of the user's face to form a composite image showing the user virtually wearing the selected frames; and
C) provide the composite images to the user on the monitor for viewing by the user.

4. The self-service optical station of claim 3, each of the frames in the frame inventory is associated with information regarding the face shape and lifestyle with which the frame is suited.

5. The self-service optical station of claim 1, further comprising at least one user input device coupled to the Elevation Control Module, wherein the Elevator Control Module adjusts the height position of the self-refractor to the eye level of the user in response to the input by the user provided through the at least one user interface device.

6. The self-service optical station of claim 2, wherein the processor comprises a Telemedicine Module configured to establish communication between the self-service optical station and a refractionist that interacts with the user either synchronously or asynchronously.

7. The self-service optical station of claim 3, wherein the Virtual Try-On Module further comprises a Measurement Module coupled to the Face Shape Analyzer Module and configured to receive the composite image of the user virtually wearing a virtual frame and to measure the user's pupil distance and segment height from the composite image.

8. The self-service optical station of claim 7, wherein the Measurement Module is configured produce a calibration factor.

9. The self-service optical station of claim 8, wherein the Measurement Module produces the calibration factor using a mark on a floor, the marking indicating where the user is to stand while the Measurement Module performs a measurement calibration procedure to produce the calibration factor.

10. The self-service optical station of claim 7, wherein:
a) the Measurement Module is further configured to measure at least one of the frame parameters from the composite image, the parameters comprising at least the frame's lens opening width, height of the lens opening, effective diameter and temple length/bridge width, the frame parameters being measured from the virtual frame, and further configured to compare the measured frame parameters to values provided by a frame manufacturer; and
b) the Virtual Frame Try-On Module is further configured to adjust the frame size of the virtual frame to match the physical dimensions provided by the frame manufacturer.

11. The self-service optical station of claim 10, wherein the Measurement Module is further configured to:
a) provide an audible instruction through the speaker 180 to instruct the user to turn the user's head to show the side of the user's face and one ear;
b) measure the distance from a corner of a hinge of the virtual frame to the user's ear;
c) use the measurement of the distance from the corner of the hinge to adjust a temple of a physical frame to be delivered to the user, such that the physical frame sits properly over the user's ear; and
d) measure the temple length of the virtual frame of the composite image wherein the length of the virtual frame is used to adjust the temple of the physical frame, to provide a better fit of the physical frame to the user's ear.

12. The self-service optical station of claim 1, the user interface devices comprising at least one of the following:
a) a touch-sensitive display screen for entering user information and interacting with the refraction control unit;
b) an eyepiece optically coupled to the self-refractor providing an optical entrance for a user's eye;
c) a headrest, where the use rests the user's head while taking a self-refraction procedure, the headrest aids in aligning the user's eye to the optical entrance of the refractor, and blocks stray light from entering the refractor;
d) a printer for printing results of an eye test;
e) a chin rest for resting a user's chin wherein the chin rest stabilizes the user's eye position during the eye tests;
f) a microphone that accepts verbal responses from the user;
g) a speaker that provides voice instructions to the user; and
h) a joystick for providing directional inputs and responses from the user, while performing subjective self-refraction procedure.

13. The self-service optical station of claim 1, further comprising a wireless fidelity (WiFi) communication network providing internet connection and coupled to the self-refractor to transmit refraction results from the self-refractor to a remote location using the WiFi network.

14. A method for providing self-service optical transactions to a user, comprising:
a) providing at a designated location, a kiosk having a pedestal and an enclosure supported by the pedestal, the enclosure containing a self-refractor;
b) moving the self-refractor to the eye level of the user;
c) performing a subjective self-refraction procedure using the self-refractor;
d) presenting at least one composite image of the user virtually wearing at least one of a subset of available frames;
e) receiving input from the user indicating which frame the user selects from among the subset of available frames;
f) selecting lenses based on the frame the user selects;
g) receiving payment from the user for the purchase of eyeglasses comprising the selected frames, and the lenses; and
h) sending a completed pair of eyeglasses to the user, wherein the completed pair of eyeglasses comprise the selected lenses inserted to the selected frame.

15. The method of claim 14, further comprising:
a) providing audible instructions to the user that guide the user through the subjective self-refraction procedure;
b) receiving responses from the user; and
c) in response to the received responses from the user, performing the subjective self-refraction procedure.

16. The method of claim 15, further comprising receiving signals from the user using a joystick, indicating the user's response to the voice instructions.

17. The method of claim 14, wherein the self-refractor provides 20 feet (or 6 meters) of optical distance.

18. The method of claim 16, further comprising performing on the user, at least one of the following:
a) an auto-refraction comprising capturing wavefront data of the user's eye;
b) a duochrome test;
c) presenting at least two choices of optical powers, while user looking at a refraction viewing target and instructing the user to choose which one of the optical presentations appears to be clearer to the user;
d) performing procedures controlling over-minuses, and
e) presenting eyecharts comprising Snellen letters, tumbling E letters, or a PSF refraction target, in visual acuity tests.

19. The method of claim 16, wherein the kiosk has a chinrest to support the user's head and stabilize the user's eye from movement and a camera, the method further comprising activating the camera in response to input from the user.

20. The method of claim 16, wherein the kiosk has a chinrest to support the user's head and stabilize the user's eye from movement, a processor and a camera, the method further comprising activating the camera in response to input from the processor.

21. The method of claim 20, further comprising;
a) receiving an image of a user's face from the camera;
b) analyzing the user's face to determine a face shape using a Face Shape Analyzer Module;
c) selecting frames from a frame database based on the determined face shape;
d) combining the image of at least one selected frame with the image of the user's face to form a composite image to show the user virtually wearing the selected virtual frame; and
e) providing the composite image to a user on a display monitor.

22. The method of claim 20, further comprising:
a) receiving information from the user about the user's lifestyle preferences;
b) selecting frames from the frame inventory based on the received information about the user's lifestyle;
c) combining the image of the selected frames with the image of the user's face to form a composite image that shows the user virtually wearing the selected virtual frame; and
d) providing the composite images on a display monitor for viewing by the user.

23. The method of claim 16, further comprising adjusting the height of the self-refractor by an elevator control module, moving the self-refractor to the eye level of the user, wherein the eye level is based on user's height information provided by the user.

24. The method of claim 16, further comprising communicating with a refractionist at a remote location using a telemedicine module, through a communicating network, wherein the refractionist receives user information including refraction results and patient in-take medical forms filled out by the user, the refractionist reviews the information and in response to receiving the information, a) approves, b) rejects or c) modifies then approves the results of the subjective self-refraction procedure.

25. The method of claim 24, wherein the reviewing process may be synchronous, or asynchronous.

26. The method of claim 23, moving the camera's position by the elevator control module to the eye level of the user, wherein a frontal image of the user's face is captured by the camera.

27. The method of claim 22, further comprising maintaining the virtual frame on the user's face as if a physical frame is being worn by the user as the moves.

28. The method of claim 22, further comprising:
a) measuring dimensions of an object in an image captured by the camera; and
b) taking an image of a credit card, wherein a calibration factor is generated based on known dimensions of the credit card and a number of pixels across the credit card in the image.

29. The method of claim 28, further comprising determining the physical dimensions of an object in the image based on a pixel number across the object and the calibration factor, the physical dimension of the object being in units of millimeters or inches.

30. The method of claim 29, placing the user on a mark on the floor in front of the station, wherein the mark selected from the group comprising: a circle, the shape of a foot, the shape of a pair of shoes, and a line,
wherein the calibration factor is generated one time for several users, wherein subsequent users stand over the marking, and wherein the generated calibration factor is used to determine distances of objects in the composite image of the user.

31. The method of claim 29, further comprising:
a) measuring frame parameters from the composite image, the frame parameters including lens opening width at widest point, the height of the lens opening, the effective diameter, temple length, bridge width, and the customer's pupil distance and segment height;
b) comparing the measured frame parameters to corresponding frame parameters of a physical frame provided by a frame manufacturer; and
c) adjusting the virtual frame size to match the frame parameters of the physical frame.

32. The method of claim 31, further comprising:
a) measuring the temple length of the virtual frame worn by the user;
b) generating voice commands requesting the user to turn the user's head 90 degrees to show the side of the user's face and the user's ear;
c) determining the distance from the virtual frame's hinge at a corner of the virtual frame to the user's ear based on the calibration factor; and
d) using the measured distance to adjust the temple of the physical frame prior to sending it to the user.

33. The method of claim 16, further comprising selecting to be displayed on a display monitor from the group comprising:
a) an image of a qualified refractionist at a remote location, the image being displayed in real time during a synchronous telemedicine session;
b) training instructions comprising video and slides showing the user what to expect in a self-refraction procedure, and the proper way to respond to certain instructions during the self-refraction procedure using interface devices; and
c) a slide presentation explaining services offered at the station.

34. The method of claim 16, further comprising providing services to, and accommodating a handicapped person in wheelchair, comprising:
a) accepting arms of a wheelchair under a base of an enclosure wherein a self-refractor is housed;
b) supporting the station with a triangular shape pedestal
c) placing the legs of a handicapped user in wheelchair under the enclosure such that the user's legs straddle the triangular support such that the user is close to the self-refractor;
d) moving the optical entrance of the refractor to the eye level of the user; and
e) moving a touch screen and at least one interface device to a height at which the user can reach and use the touch screen and interface device.

35. The method of claims 22, further comprising:
a) ordering prescription lenses for making an eyeglass; and
b) accepting payment for a purchase of the selected frames and lenses.

36. The method of claim 35, transmitting the purchase order through a communication network to a fabrication facility to manufacture a complete eyeglass, comprising assembling selected lenses in the selected frames.
